(19) United States
(12) Patent Application Publication
(10) Pub. No.: US 2007/0082019 A1
(43) Pub. Date: Apr. 12, 2007

(54) Photocrosslinked hydrogel surface coatings

(75) Inventors: Wenxi Huang, Fremont, CA (US); Yury Agroskin, Cupertino, CA (US); Egisto Boschetti, Crossy sur Seine (FR)

Correspondence Address: CIPHERGEN c/o FOLEY & LARDNER LLP, 3000 K STREET NW, SUITE 500, WASHINGTON, DC, 20007, US

(73) Assignee: CIPHERGEN BIOSYSTEMS INC.

(21) Appl. No.: 10/546,173

(22) PCT Filed: Feb. 20, 2004
(86) PCT No.: PCT/US04/04847

§ 371 Date: Oct. 24, 2006

Related U.S. Application Data
(60) Provisional application No. 60/448,467, filed on Feb. 21, 2003.

(51) Int. Cl. A61F 2/02; C12M 1/34

(52) U.S. Cl. 424/423; 435/287.2; 427/2.11; 427/2.24

(57) ABSTRACT

A hydrogel layer is applied to a substrate advantageously when the layer is formed in situ, using a polymeric or copolymeric precursor that includes, respectively, monomer subunits that have a photocrosslinkable functionality and monomer subunits that have a chemically selective functionality for binding a biomolecular analyte, such as a protein. A hydrogel-coated substrate thus obtained is particularly useful as a probe for mass spectroscopic analysis, including SELDI analysis. Hydrogel particles also can be used for SELDI analysis.

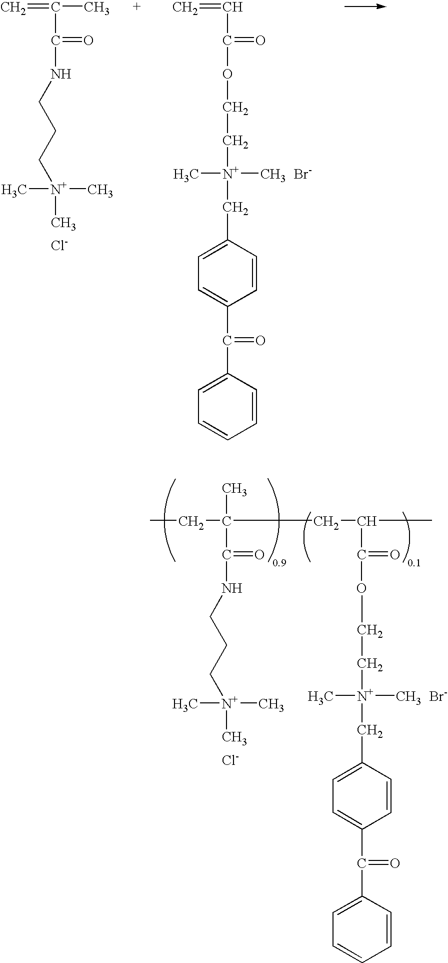

### PRIORITY

This application claims priority to a U.S. provisional application Ser. No. 60/448,467, that was filed on Feb. 21, 2003 to Huang et al., and that is entitled “Photocrosslinked Hydrogel Surface Coatings.”

### BACKGROUND

The term “hydrogel” generally connotes a hydrophilic, crosslinked, organic polymeric material (i.e., hydrophilic polymer networks) that swells in and retains water (see, e.g., WO00/66265 to Ciphergen Biosystems). Hydrogels have a variety of commercial applications, illustrated by their use in contact lens, sensors, tissue adhesives, drug delivery, dressings, and surface coatings. For example, see U.S. Pat. No. 6,017,577 to Hostettler et al. In particular, hydrogel surface coatings are used in biomedical devices, such as catheters, catheter balloons, and stents, as illustrated by U.S. Pat. No. 5,601,538 to Deem. Hydrogels can be applied as continuous layers or as patterns of discreet regions on a surface (e.g., gel “patches” or “pads”).

The distinctive ability of hydrogels to swell extensively in water, forming a structurally stable but liquid-compatible structure, arises from their lightly crosslinked character, which in turn arises from how they are made. One approach to such manufacture is by photopolymerization and photocrosslinking, respectively, as disclosed in U.S. Pat. No. 5,567,435 and No. 6,156,478. Thus, the '478 patent describes photocrosslinkable and photopatternable hydrogel compositions that are based on an azlactone-functional monomer. These hydrogels can be patterned onto a substrate by means of a photomask or laser-induced thermal imaging, and the azlactone functionality can be used to bind biomolecules to the substrate. According to the '478 patent, the described hydrogel compositions can be used to produce a “microchip,” such as a low- or high-density DNA chip or a microarray of enzyme-containing gel pads.

Another approach to producing hydrogel materials is by deposition of a monomer solution on the substrate surface and in situ polymerization and crosslinking of monomer mixture using a thermal or photoinitiator, as disclosed in PCT application WO 00/66265. Changing the amount of monomer and cross-linker can affect the thickness and pore size of the resulting hydrogel layers.

U.S. Pat. Nos. 5,512,329 and 5,002,582 to Guire et al. discloses polymers which have latent reactive groups for covalent bonding to substrate surfaces. These polymers covalently bond to the substrate surface when the latent reactive groups are stimulated by an external stimulus such as actinic radiation. These polymers, however, are generally designed for repelling protein rather than adsorbing proteins, or selectively interacting with and binding of proteins with tailored control of functional group chemistry. Moreover, these polymers are not prepared by controlled copolymerization methods which allow for suitable hydrogel formation and suitable chemical selectivity with proteins and other biomolecules.

Despite their demonstrated versatility and applicability in certain contexts, the potential of hydrogels has not been fully exploited in mass-spectral techniques, such as Matrix-Assisted Laser Desorption/Ionization (MALDI) and Surface-Enhanced Laser Desorption/Ionization (SELDI) mass spectroscopy, which are of increasing popularity for protein analysis. Moreover, conventional procedures for producing hydrogels typically do not provide the coating uniformity and homogeneity that would facilitate MALDI or SELDI mass spectroscopy. For example, using in situ polymerization of monomer mixture do not typically provide controlled polymerization processes. The polymerization and surface attachment typically take place simultaneously on an individual spot, and each spot represents a separated reactor. The resulting hydrogel materials can suffer from spot-to-spot and chip-to-chip variations. The conventional procedures also typically do not provide a three-dimensional polymeric structure that has sufficient surface area, controllable porosity and ligand density for capturing proteins and biomolecules in a broad range of molecular weight. The hydrogels having sufficient surface area can provide a probe with high binding capacity and sensitivity, which is attractive when the amount of the sample available for analysis is very small and limited. The hydrogels having controllable pore size and/or ligand density can provide a probe with desirable selectivity and binding capacity that meet the demands of specific biological applications. Also, conventional methods typically do not provide the coating uniformity and homogeneity that would facilitate MALDI or SELDI mass spectroscopy. For instance, uniformity in the hydrogel surface coating may provide a more accurate time-of-flight analysis of samples, as all analytes absorbed on the probe surface are equidistant from an energy source of a gas phase ion spectrometer. Also, conventional formulations for probe materials may not be compatible with desired process methods such as spin coating, dip coating, photopatterning, or useful combinations thereof. The presence of low molecular weight components can cause problems. For instance, see PCT application WO 00/66265 and U.S. patent publication 20020060290 A1.

A need exists to improve MALDI, SELDI, and other mass-spectrometric analyses through use of probe materials characterized by greater uniformity and structural stability, through better control of coating thickness, hydrogel porosity, and spot variation. Advantages which the present invention provides include maximizing the value of a hydrogel surface for SELDI and MALDI analysis including but not limited to the following factors: (1) complete coverage of the hydrogel, (2) control of hydrogel thickness and swelling degree, (3) uniformity of hydrogel coatings, (4) stability of hydrogel on the surface, (5) controlling the density of the chemically selective, binding functionality, (6) ease and consistency of producing hydrogel, and (7) substantially absence of low molecular weight components which can diffuse out and interfere with the analyses by generating signal noise.

### SUMMARY OF THE INVENTION

To satisfy these needs and others, the present invention provides, according to one preferred embodiment, a copolymeric hydrogel precursor comprising: (a) first monomeric subunits that comprise a photocrosslinkable functionality and (b) second monomeric subunits that comprise a chemically selective functionality for binding a protein, wherein the amounts of first and second monomeric subunits provide the copolymeric hydrogel precursor with the ability to be photocrosslinked into a hydrogel and the ability for the hydrogel to be selectively bind with protein under aqueous conditions, whereby protein becomes bound to the chemically selective functionality.

The present invention also includes an embodiment comprising a polymeric hydrogel precursor comprising photocrosslinkable functionality and chemically selective functionality, wherein the precursor is prepared by functionalizing a prefunctionalized polymeric hydrogel precursor with photocrosslinkable functionality and with chemically selective functionality, wherein the amounts of photocrosslinkable functionality and chemically selective functionality provide the hydrogel precursor with the ability to be photocrosslinked into the hydrogel and the ability for the hydrogel to be selectively bound with protein under aqueous conditions, whereby protein becomes bound to the chemically selective functionality.

Another embodiment is a copolymeric hydrogel precursor prepared by copolymerization of monomeric subunits comprising:

(a) first monomeric subunits that comprise a first free radical copolymerization functionality and a photocrosslinkable functionality and

(b) second monomeric subunits that comprise a second free radical copolymerization functionality and a chemically selective functionality for binding a protein,

wherein the amounts of first and second monomeric subunits provide, upon copolymerization, the polymeric hydrogel precursor with the ability to be photocrosslinked into the hydrogel and the ability to be selectively reactive with protein under aqueous conditions, whereby protein becomes bound to the chemically selective functionality.

Also provided is an embodiment for a photocrosslinkable hydrogel precursor composition for selective interaction with protein under aqueous conditions consisting essentially of at least one hydrogel precursor polymer consisting essentially of (i) first comonomeric subunits that comprise a photocrosslinkable functionality and (ii) second comonomeric subunits that comprise a chemically selective functionality for interaction with a biomolecular analyte, wherein the photocrosslinkable hydrogel precursor composition is substantially free of photoinitiator.

According to another aspect of the present invention, a method is provided for functionalizing a surface with copolymeric hydrogel, comprising:
(A) providing (i) a substrate presenting a surface and (ii) a copolymeric hydrogel precursor that comprises (a) first comonomeric subunits that comprise a photocrosslinkable functionality and (b) second comonomeric subunits that comprise a chemically selective functionality for binding a biomolecular analyte;
(B) contacting the copolymeric hydrogel precursor and the surface to form a layer of the copolymeric hydrogel precursor on the surface; and
(C) photocrosslinking at least some of the copolymeric hydrogel precursor layer to form hydrogel in contact with the surface.

Still further, the present invention provides a substrate that comprises a substrate surface and a hydrogel thereon, wherein the hydrogel comprises (i) a photocrosslinked functionality and (ii) a chemically selective functionality for binding a biomolecular analyte, wherein the hydrogel is substantially free of photoinitiator, and wherein the amount of chemically selective functionality is sufficient for binding the biomolecular analyte (i.e., “the substrate according to the present invention”).

The invention also provides a method of detecting an analyte, comprising (a) contacting the substrate according to the present invention with a sample that contains a biomolecular analyte and then (b) detecting the biomolecular analyte by virtue of its binding said chemically selective functionality, in particular by laser desorption mass spectrometry.

As a result, better analyses, including laser desorption/ionization mass spectrometry analyses, can be achieved over more diverse systems. Advantages of the photocrosslinking process include mild conditions, minimum side-product formation, fast cure times, and spatial control of the crosslinking reaction. Also, the physiochemical properties of the polymer network such as, for example, swelling can be modulated by adjusting illumination and concentration of the photocrosslinkable group. The chemical formulation is applicable to a variety of process methods. Other considerations include without limitation the invention providing: (1) three-dimensional polymeric hydrogels with sufficient surface area, resulting in increased binding capacity along with marked improvement in binding selectivity; (2) improved control of the cross-linking reaction, thus resulting in a more uniform hydrogel with desired pore size suitable for capturing proteins and biomolecules in a broad range of molecular weight; (3) a polymerization process used to produce polymers which is more consistent and controllable, and use of polymers instead of monomers which provide sufficient viscosity that is more compatible with established processing methods and improves chip manufacturing; (4) producing polymers in bulk allows one to form uniform and consistent coating surface essentially eliminating variations, both spot-to-spot and chip-to-chip in material composition and film thickness; (5) better and more complete coverage of the hydrogel surface reducing non-specific binding which can affect capturing of analytes and generate signal noise in the mass analysis step; (6) hydrogel materials having greater structural stability, resulting in improved duration life time and consistent sample capturing.

Finally, the invention also provides for a copolymeric hydrogel precursor comprising:

(a) first monomeric subunits that comprise a photocrosslinkable functionality, and

(b) third monomeric subunits that comprise an energy absorbing moiety.

Here, the hydrogel can further comprise second monomeric subunits that comprise a chemically selective functionality for binding a protein or other biomolecular analyte, which can comprise a covalently binding moiety or a non-covalent binding moiety.

### BRIEF DESCRIPTION OF THE DRAWINGS

FIG. 1. Preparation of Copolymer of SAX Monomer and Photocrosslinkable Monomer.

FIG. 2. Reflectance FTIR Spectrum of a CVD-methacrylate Silane Primer Layer on Aluminum Substrate.

FIG. 3. Reflectance FTIR Spectrum of SAX Hydrogel Coating on a SiO₂-coated Aluminum Substrate.

FIG. 4. Mass Spectrum at Low and High Mass of Serum Profiling.

FIG. 5. Effect of Benzophenone Concentration and Hydrogel Pore Size on SELDI Signal Intensity and Peak Count of Serum Profiling.

FIG. 6. Effect of hydrogel coating thickness on SELDI signal intensity an peak count of Serum profiling.

FIG. 7. Fluorescent Bovine Serum Albumin Bound to: (a) SAX-2 Chip Prepared by in-situ Grafting Polymerization; (b) SAX Chip Prepared by Current Inventive Method.

FIG. 8. Preparation of Monomer having photocrosslinkable functionality.

FIG. 9. Preparation of Copolymer of SAX monomer and photocrosslinkable monomer.

FIG. 10. Copolymerization of WCX monomer with monomer having photocrosslinkable functionality to prepare photocrosslinkable WCX copolymer

FIG. 11. Preparation of Monomer Having Photocrosslinkable Functionality.

FIG. 12. Copolymerization of WCX Monomer with Monomer Having Photocrosslinkable Functionality to Prepare Photocrosslinkable WCX Copolymer.

FIG. 13. Reflectance FTIR Spectrum of WCX Hydrogel Coating on a SiO₂-coated Aluminum Substrate

FIG. 14. Serum profiling of WCX Chip.

FIG. 15. H50 Copolymer

FIG. 16. Serum Fr.2 profiling of H50 Chip

FIG. 17. Fluorescent bovine serum albumin bound to H50 chips.

FIG. 18. IMAC Copolymer

FIG. 19. Illustration of Dextran Chemistry.

FIG. 20. Reflectance FTIR Spectra of (a) Benzophenone-modified Dextran Hydrogel Coating on a Aluminum Substrate; (b) CDI-activated Dextran Hydrogel Coating on a Aluminum Substrate.

FIG. 21. Preparation of N-succinimide Containing Copolymer for Pre-activated Surface.

FIG. 22. Preparation of Functional Cross-linkable Polymers via Polymer Modification

FIG. 23. Derivatization of Poly(2-hydroxyethyl methacrylate) with Photocrosslinkable Group.

FIG. 24. Approach to Prepare Uniform Polymer Coating across Whole Chip.

FIG. 25. Use of Photomask for Spot Control.

FIG. 26. Semi-conductor die-attach process for chip production.

FIG. 27. Reaction scheme for copolymer including an energy absorbing moiety.

### DETAILED DESCRIPTION OF PREFERRED EMBODIMENTS

In this application, which describes improved materials and methods of mass spectroscopy, the following terms are used:

“Gas phase ion spectrometer” refers to an apparatus that detects gas phase ions. Gas phase ion spectrometers include an ion source that supplies gas phase ions. Gas phase ion spectrometers include, for example, mass spectrometers, ion mobility spectrometers, and total ion current measuring devices.

“Gas phase ion spectrometry” refers to the use of a gas phase ion spectrometer to detect gas phase ions.

“Mass spectrometer” refers to a gas phase ion spectrometer that measures a parameter which can be translated into mass-to-charge ratios of gas phase ions. Mass spectrometers generally include an ion source and a mass analyzer. Examples of mass spectrometers are time-of-flight, magnetic sector, quadrupole filter, ion trap, ion cyclotron resonance, electrostatic sector analyzer and hybrids of these.

“Mass spectrometry” refers to the use of a mass spectrometer to detect gas phase ions.

“Laser desorption mass spectrometer” refers to a mass spectrometer which uses laser as a means to desorb, volatilize, and ionize an analyte.

“Tandem mass spectrometer” refers to any mass spectrometer that is capable of performing two successive stages of m/z-based discrimination or measurement of ions, including of ions in an ion mixture. The phrase includes mass spectrometers having two mass analyzers that are capable of performing two successive stages of m/z-based discrimination or measurement of ions tandem-in-space. The phrase further includes mass spectrometers having a single mass analyzer that are capable of performing two successive stages of m/z-based discrimination or measurement of ions tandem-in-time. The phrase thus explicitly includes Qq-TOF mass spectrometers, ion rap mass spectrometers, ion trap-TOF mass spectrometers, TOF-TOF mass spectrometers, and Fourier transform ion cyclotron resonance mass spectrometers, electrostatic sector—magnetic sector mass spectrometers, and combinations thereof.

“Mass analyzer” refers to a sub-assembly of a mass spectrometer that comprises means for measuring a parameter which can be translated into mass-to-charge ratios of gas phase ions. In a time-of flight mass spectrometer the mass analyzer comprises an ion optic assembly, a flight tube and an ion detector.

“Ion source” refers to a sub-assembly of a gas phase ion spectrometer that provides gas phase ions. In one embodiment, the ion source provides ions through a desorption/ionization process. Such embodiments generally comprise a probe interface that positionally engages a probe in an interrogatable relationship to a source of ionizing energy (e.g., a laser desorption/ionization source) and in concurrent communication at atmospheric or subatmospheric pressure with a detector of a gas phase ion spectrometer.

Illustrative of the forms of ionizing energy for desorbing/ionizing an analyte from a solid phase are: (1) laser energy; (2) fast atoms (used in fast atom bombardment); (3) high energy particles generated via beta decay of radionucleides (used in plasma desorption); and (4) primary ions generating secondary ions (used in secondary ion mass spectrometry). The preferred form of ionizing energy for solid phase analytes is a laser (used in laser desorption/ionization), in particular, nitrogen lasers, Nd-Yag lasers, and other pulsed laser sources. Other forms of ionizing energy for analytes include, for example: (1) electrons which ionize gas phase neutrals; (2) strong electric field to induce ionization from gas phase, solid phase, or liquid phase neutrals; and (3) a source that applies a combination of ionization particles or electric fields with neutral chemicals to induce chemical ionization of solid phase, gas phase, and liquid phase neutrals.

“Fluence” refers to the laser energy delivered per unit area of interrogated image. A high fluence source, such as a laser, will deliver about 1 mJ/mm² to 50 mJ/mm². Typically, a sample is placed on the surface of a probe, the probe is engaged with the probe interface and the probe surface is struck with the ionizing energy. The energy desorbs analyte molecules from the surface into the gas phase and ionizes them.

“Probe” in the context of this invention refers to a device adapted to engage a probe interface and to present an analyte to ionizing energy for ionization and introduction into a gas phase ion spectrometer, such as a mass spectrometer. A “probe” will generally comprise a solid substrate (either flexible or rigid) comprising a sample presenting surface on which an analyte is presented to the source of ionizing energy.

“Surface-enhanced laser desorption/ionization” or “SELDI” refers to a method of desorption/ionization gas phase ion spectrometry (e.g., mass spectrometry) in which the analyte is captured on the surface of a SELDI probe that engages the probe interface of the gas phase ion spectrometer. In “SELDI MS,” the gas phase ion spectrometer is a mass spectrometer. SELDI technology is described in, e.g., U.S. Pat. No. 5,719,060 (Hutchens and Yip) and U.S. Pat. No. 6,225,047 (Hutchens and Yip).

“Surface-Enhanced Affinity Capture” or “SEAC” is a version of SELDI that involves the use of probes comprising an absorbent surface (a “SEAC probe”). “Adsorbent surface” refers to a surface to which is bound an adsorbent (also called a “capture reagent” or an “affinity reagent”). An adsorbent is any material capable of binding an analyte (e.g., a target polypeptide or nucleic acid). “Chromatographic adsorbent” refers to a material typically used in chromatography. Chromatographic adsorbents include, for example, ion exchange materials, metal chelators (e.g., nitriloacetic acid or iminodiacetic acid), immobilized metal chelates, hydrophobic interaction adsorbents, hydrophilic interaction adsorbents, dyes, simple biomolecules (e.g., nucleotides, amino acids, simple sugars and fatty acids) and mixed mode adsorbents (e.g., hydrophobic attraction/electrostatic repulsion adsorbents). “Biospecific adsorbent” refers an adsorbent comprising a biomolecule, e.g., a nucleic acid molecule (e.g., an aptamer), a polypeptide, a polysaccharide, a lipid, a steroid or a conjugate of these (e.g., a glycoprotein, a lipoprotein, a glycolipid). In certain instances the biospecific adsorbent can be a macromolecular structure such as a multiprotein complex, a biological membrane or a virus. Examples of biospecific adsorbents are antibodies, receptor proteins and nucleic acids. Biospecific adsorbents typically have higher specificity for a target analyte than chromatographic adsorbents. Further examples of adsorbents for use in SELDI can be found in U.S. Pat. No. 6,225,047 (Hutchens and Yip, “Use of retentate chromatography to generate difference maps,” May 1, 2001).

In some embodiments, a SEAC probe is provided as a pre-activated surface which can be modified to provide an adsorbent of choice. For example, certain probes are provided with a reactive moiety that is capable of binding a biological molecule through a covalent bond. Epoxide and carbodiimidizole are useful reactive moieties to covalently bind biospecific adsorbents such as antibodies or cellular receptors.

“Adsorption” refers to detectable non-covalent binding of an analyte to an adsorbent or capture reagent.

“Surface-Enhanced Neat Desorption” or “SEND” is a version of SELDI that involves the use of probes comprising energy absorbing molecules chemically bound to the probe surface. (“SEND probe.”) “Energy absorbing molecules” (“EAM”) refer to molecules that are capable of absorbing energy from a laser desorption/ionization source and thereafter contributing to desorption and ionization of analyte molecules in contact therewith. The phrase includes molecules used in MALDI, frequently referred to as “matrix”, and explicitly includes cinnamic acid derivatives, sinapinic acid (“SPA”), cyano-hydroxy-cinnamic acid (“CHCA”) and dihydroxybenzoic acid, ferulic acid, hydroxyacetophenone derivatives, as well as others. It also includes EAMs used in SELDI. SEND is further described in U.S. Pat. No. 5,719,060. Use of EAMs in the present hydrogels is described further below.

“Surface-Enhanced Photolabile Attachment and Release” or “SEPAR” is a version of SELDI that involves the use of probes having moieties attached to the surface that can covalently bind an analyte, and then release the analyte through breaking a photolabile bond in the moiety after exposure to light, e.g., laser light. SEPAR is further described in U.S. Pat. No. 5,719,060.

“Adsorption” refers to detectable non-covalent binding of an analyte to an adsorbent or capture reagent.

“Eluant” or “wash solution” refers to an agent, typically a solution, which is used to affect or modify adsorption of an analyte to an adsorbent surface and/or remove unbound materials from the surface. The elution characteristics of an eluant can depend, for example, on pH, ionic strength, hydrophobicity, degree of chaotropism, detergent strength and temperature.

“Analyte” refers to any component of a sample that is desired to be detected. The term can refer to a single component or a plurality of components in the sample.

The “complexity” of a sample adsorbed to an adsorption surface of an affinity capture probe means the number of different protein species that are adsorbed.

“Molecular binding partners” and “specific binding partners” refer to pairs of molecules, typically pairs of biomolecules that exhibit specific binding. Molecular binding partners include, without limitation, receptor and ligand, antibody and antigen, biotin and avidin, and biotin and streptavidin.

“Monitoring” refers to recording changes in a continuously varying parameter.

“Biochip” refers to a solid substrate having a generally planar surface to which a capture reagent (adsorbent) is attached. Frequently, the surface of the biochip comprises a plurality of addressable locations, each of which location has the capture reagent bound there. Biochips can be adapted to engage a probe interface and, therefore, function as probes.

“Protein biochip” refers to a biochip adapted for the capture of polypeptides. Many protein biochips are described in the art. These include, for example, protein biochips produced by Ciphergen Biosystems (Fremont, Calif.), Packard BioScience Company (Meriden Conn.), Zyomyx (Hayward, Calif.), Phylos (Lexington, Mass.) and Procognia (Sense Proteomic Limited) (Maidenhead, Berkshire, UK). Examples of such protein biochips are described in the following patents or patent applications: U.S. Pat. No. 6,225,047 (Hutchens and Yip, “Use of retentate chromatography to generate difference maps,” May 1, 2001); International publication WO 99/51773 (Kuimelis and Wagner, “Addressable protein arrays,” Oct. 14, 1999); U.S. Pat. No. 6,329,209 (Wagner et al., “Arrays of protein-capture agents and methods of use thereof,” Dec. 11, 2001), International publication WO 00/56934 (Englert et al., “Continuous porous matrix arrays,” Sep. 28, 2000), United States patent publication US 2003/0180957 A1 (Koopman et al., “Target and method,” Sep. 25, 2003) and United States patent publication US 2003/0173513 A1 (Koopman et al., “Probe for mass spectrometry,” Sep. 18, 2003).

Protein biochips produced by Ciphergen Biosystems comprise interacting surfaces having chromatographic or biospecific adsorbents attached thereto at addressable locations. Ciphergen ProteinChip® arrays include NP20, H4, H50, SAX-2, WCX-2, IMAC-3, LSAX-30, LWCX-30, IMAC-40, PS-10, PS-20 and PG-20. These protein biochips can comprise an aluminum substrate in the form of a strip. The surface of the strip can be coated with silicon dioxide. In the case of the NP-20 biochip, silicon oxide functions as a hydrophilic adsorbent to capture hydrophilic proteins.

H4, H50, SAX-2, WCX-2, IMAC-3, PS-10 and PS-20 biochips further comprise a functionalized, cross-linked polymer in the form of a hydrogel physically attached to the surface of the biochip or covalently attached through a silane to the surface of the biochip. The H4 biochip has isopropyl functionalities for hydrophobic binding. The H50 biochip has nonylphenoxy-poly(ethylene glycol)methacrylate for hydrophobic binding. The SAX-2 biochip has quaternary ammonium functionalities for anion exchange. The WCX-2 biochip has carboxylate functionalities for cation exchange. The IMAC-3 biochip has nitriloacetic acid functionalities that adsorb transition metal ions, such as Cu⁺⁺ and Ni⁺⁺, by chelation. These immobilized metal ions allow adsorption of peptide and proteins by coordinate bonding. The PS-10 biochip has carboimidizole functional groups that can react with groups on proteins for covalent binding. The PS-20 biochip has epoxide functional groups for covalent binding with proteins. The PS-series biochips are useful for binding biospecific adsorbents, such as antibodies, receptors, lectins, heparin, Protein A, biotin/streptavidin and the like, to chip surfaces where they function to specifically capture analytes from a sample. The PG-20 biochip is a PS-20 chip to which Protein G is attached. The LSAX-30 (anion exchange), LWCX-30 (cation exchange) and IMAC-40 (metal chelate) biochips have functionalized latex beads on their surfaces. Such biochips are further described in WO 00/66265 (Rich et al., “Probes for a Gas Phase Ion Spectrometer,” Nov. 9, 2000) and in WO 00/67293 (Beecher et al., “Sample Holder with Hydrophobic Coating for Gas Phase Mass Spectrometer,” Nov. 9, 2000).

Upon capture on a biochip, analytes can be detected by a variety of detection methods including for example, gas phase ion spectrometry methods, optical methods, electrochemical methods, atomic force microscopy and radio frequency methods. Gas phase ion spectrometry methods are described herein. Of particular interest is the use of mass spectrometry and, in particular, SELDI. Optical methods include, for example, detection of fluorescence, luminescence, chemiluminescence, absorbance, reflectance, transmittance, birefringence or refractive index (e.g., surface plasmon resonance, ellipsometry, a resonant mirror method, a grating coupler waveguide method or interferometry). Optical methods include microscopy (both confocal and non-confocal), imaging methods and non-imaging methods. Immunoassays in various formats (e.g., ELISA) are popular methods for detection of analytes captured on a solid phase. Electrochemical methods include voltametry and amperometry methods. Radio frequency methods include multipolar resonance spectroscopy.

I. Polymers and Compositions

In the present invention, analytical methods such as the MALDI and SELDI methods described above, can be improved by use of novel hydrogel compositions. These compositions can be prepared by novel copolymeric hydrogel precursor compositions which are subjected to photocrosslinking.

The structural type of the copolymeric hydrogel precursor and the type of polymeric backbone is not particularly limited but can be, for example, a linear polymer, a branched polymer, or a dendritic polymer. Although the copolymeric hydrogel precursor is not generally crosslinked prior to conversion to the hydrogel under photocrosslinking conditions, it nevertheless may be in some cases water-swellable only and not water soluble. In other words, the copolymeric hydrogel precursor can be water-soluble or water-swellable. Monomers and prepolymers can be used which are known in the art to provide hydrogels. Preferably, the copolymeric hydrogel precursor comprises a linear polymeric backbone that is comprised of carbon and that carries first side groups having the photocrosslinkable functionality and second side groups having the chemically selective functionality.

The copolymeric hydrogel precursor can be prepared by a variety of synthetic methods which are not particularly limited. Upon synthesis, the copolymeric hydrogel precursor can be a polymer chain having a polymeric backbone and at least two kinds of functionality covalently bound to the polymer backbone: photocrosslinkable functionality and chemically selective functionalities. Chemically selective functionalities are able to select preferentially a target based on chemical interactions known in the art including, for example, covalent binding, non-covalent binding, electrostatic binding, and other modes described further herein. These functionalities can be regularly or randomly distributed along the polymeric backbone. Random distribution can help improve uniformity in the polymeric material, which can aid in the accuracy of the mass spectroscopic applications such as SELDI. Upon crosslinking of the hydrogel, the chemically selective functionalities can selectively react with proteins and other biomolecular analytes and targets including covalent and non-covalent binding reaction.

In preferred embodiments, the copolymeric hydrogel precursor is a dextran or polyolefin composition. In a preferred embodiment, the copolymeric hydrogel precursor comprises a linear, carbon backbone represented by monomeric subunits:

—(CH₂—CHR₁)— and —CH₂—CHR₂)—, —[CH₂—C(CH₃)R₁]— and —[CH₂—C(CH₃)R₂]—;

wherein R₁ and R₂ comprise the groups with the photocrosslinkable and chemically selective functionalities, respectively. In general, monomers provide either the photocrosslinkable functionality or the chemically selective functionalities. However, other monomers can be present in the polymeric hydrogel precursor as well. The presence of these monomers can be used to control the density of binding functionalities desired in the polymer composition. Suitable selection of monomers can provide the resulting polymeric hydrogel precursor with improved water-solubility, biocompatibility, and with reduced non-specific absorption. Preferred monomers provide an optimal combination of such properties. The copolymeric hydrogel precursor, in preferred embodiments, consists essentially of a linear copolymeric backbone having side groups that comprise the photocrosslinkable functionality and the chemically selective functionality, wherein other structural units in the copolymer do not interfere with the ability to be photocrosslinked and, upon crosslinking, to selectively react with protein under aqueous conditions. In general, the copolymer structure is designed to bind selectively proteins, not repel proteins.

The first monomeric subunits that comprise a photocrosslinkable functionality are not particularly limited. These first monomeric subunits can function as a photoinitiator in a photocrosslinkable polymeric composition. In other words, because of these monomer subunits, photoinitiator does not need to be, and preferably is not, added to the composition for photocrosslinking. For example, the photocrosslinkable functionality can be a UV-curable functionality. The photocrosslinkable functionality is sufficiently sensitive to photons that it becomes highly reactive when exposed to photocrosslinking conditions so that a photoinitiator is not needed to generate photocrosslinking. For example, the photocrosslinkable functionality can be capable of hydrogen abstraction reactions when exposed to photocrosslinking conditions. Examples of photocrosslinkable functionalities include benzophenone, diazo ester, aryl azide, and diazirine, including derivatives thereof such as benzophenone derivatives. Hydrogen abstraction chemistry for benzophenone type compounds is disclosed, for example, in U.S. Pat. No. 5,856,066. Additional photocrosslinkable functionalities, or so-called “latent reactive groups,” are described in for example U.S. Pat. No. 5,002,582.

In a preferred embodiment, the photocrosslinkable functionality is a ketone functionality, or an organic carbonyl functionality, including for example aromatic ketone functionality such as substituted benzophenone and derivatives thereof. The carbonyl carbon can have at least one substituted or unsubstituted aromatic ring bonded to it. In a preferred embodiment, photocrosslinkable vinyl monomers can be used. Acrylate, methacrylate, acrylamide, and methacrylamide systems are preferred embodiments. Traditional coupling reactions between, for example, hydroxyl and carboxylic acid, or amino groups and carboxylic acids can be used to form monomers having photocrosslinking groups, as well as other groups described herein including chemically selective binding groups and energy absorbing moieties.

Benzophenone and its derivatives are preferred as they have several advantages including: chemical stability, activation at wavelengths such as 350-360 nm which avoid protein damage, preferential reaction with unreactive C—H bonds even in the presence of water and bulk nucleophiles.

The second monomeric subunits that comprise a chemically selective functionality for binding a biomolecular analyte, particularly protein, are not particularly limited. Selective binding and reaction can be based on covalent or non-covalent interactions and the binding moieties can be covalent binding moieties or non-covalent binding moieties. For example, a variety of chemically selective functionalities are described in U.S. patent publication No. 2002/0060290 A1 and WO 00/66265, cited above. Thus, the '290 patent publication discloses a variety of adsorbents beginning at paragraph 70 which bind analytes. These include adsorbents based on salt-promoted interactions (paragraph 73), hydrophilic interaction adsorbents (paragraph 80), electrostatic interaction adsorbents (paragraph 84), coordinate covalent interaction adsorbents (paragraph 93), enzyme-active site interaction adsorbents (paragraph 98), reversible covalent interaction adsorbents (paragraph 100), glycoprotein interaction adsorbents (paragraph 102), and biospecific interaction adsorbents (paragraph 104). Other interactions include hydrophobic interactions. Combinations of interactions can be used.

Furthermore, WO 00/66265 discloses a series of chemically selective functionalities, or binding functionalities, including those listed at pages 13-15.

Hydrogel adsorbents are the materials that bind the biomolecular analytes. As described below, adsorbents are known that can be adapted in structure to be hydrogels and to be photocrosslinkable, by virtue of photocrosslinkable functionality as described above. They are attached to the surface of the substrates that form the probes. A plurality of adsorbents can be employed in the methods of this invention. Different adsorbents can exhibit grossly different binding characteristics, somewhat different binding characteristics, or subtly different binding characteristics.

Hydrogel adsorbents that exhibit grossly different binding characteristics typically differ in their bases of attraction or mode of interaction. The basis of attraction is generally a function of chemical or biological molecular recognition. Bases for attraction between an adsorbent and an analyte include, for example, (1) a salt-promoted interaction, e.g., hydrophobic interactions, thiophilic interactions, and immobilized dye interactions; (2) hydrogen bonding and/or van der Waals forces interactions and charge transfer interactions, such as in the case of a hydrophilic interactions; (3) electrostatic interactions, such as an ionic charge interaction, particularly positive or negative ionic charge interactions; (4) the ability of the analyte to form coordinate covalent bonds (i.e., coordination complex formation) with a metal ion on the adsorbent; or combinations of two or more of the foregoing modes of interaction. That is, the adsorbent can exhibit two or more bases of attraction, and thus be known as a “mixed functionality” adsorbent.

1. Salt-promoted Interaction Hydrogel Adsorbents

Adsorbents that are useful for observing salt-promoted interactions include hydrophobic interaction adsorbents. Illustrative of hydrophobic interaction adsorbents are matrices that have aliphatic hydrocarbons, specifically C1-C18 linear or branched aliphatic hydrocarbons; and matrices that have aromatic hydrocarbon functional groups such as phenyl groups.

Another adsorbent useful for observing salt-promoted interactions includes thiophilic interaction adsorbents, such as for example T-GEL.RTM. which is one type of thiophilic adsorbent commercially available from Pierce (Rockford, Ill.). A third adsorbent category, involving salt-promoted ionic interactions and also hydrophobic interactions, includes immobilized dye interaction adsorbents. Immobilized dye interaction adsorbents include matrices of immobilized dyes such as for example Cibacron Blue F3GA available from various sorbent vendors.

a) Reverse Phase Hydrogel Adsorbent—Aliphatic Hydrocarbon One useful reverse phase adsorbent is a hydrophobic (C16) H4 chip and H50 chip, available from Ciphergen Biosystems, Inc. (Palo Alto, Calif.). The hydrophobic H4 chip comprises C16 chains immobilized on top of silicon oxide (SiO.sub.2) as the adsorbent on the substrate surface. For description of the H50 chip, one skilled in the art can review U.S. patent publication no. 2003/0124371 A1.

2. Hydrophilic Interaction Hydrogel Adsorbents

Adsorbents useful for observing hydrogen bonding and/or van der Waals forces, on the basis of hydrophilic interactions, include surfaces comprising normal phase adsorbents such as silicon-oxide (e.g., glass and other mineral oxides). Silanols and siloxanes present on normal phase or silicon-oxide surface can act as a hydrophilic interacting group. In addition, adsorbents comprising surfaces modified with hydrophilic polymers such as polyethylene glycol, dextran, agarose, or cellulose can also function as hydrophilic interaction adsorbents. Most proteins will bind hydrophilic interaction adsorbents because of a group or combination of amino acid residues (i.e., hydrophilic amino acid residues) that bind through hydrophilic interactions involving hydrogen bonding or van der Waals forces.

a) Normal Phase Hydrogel Adsorbent—Silicon Oxide

One useful hydrophilic adsorbent is a Normal Phase chip, available from Ciphergen Biosystems, Inc. (Palo Alto, Calif.). The normal phase chip comprises silicon oxide (SiO₂) as the adsorbent on the substrate surface. Silicon oxide can be applied to the surface by any of a number of well known methods. These methods include, for example, vapor deposition, e.g., sputter coating. A preferred thickness for such a probe is about 9000 Angstroms.

3. Electrostatic Interaction Hydrogel Adsorbents

Adsorbents which are useful for observing electrostatic or ionic charge interactions include anionic adsorbents such as, for example, matrices of sulfate anions (i.e., SO.sub.3.sup.-) and matrices of carboxylate anions (i.e., COO.sup.-) or phosphate anions (OPO.sub.3.sup.-). Matrices having sulfate anions are permanent negatively charged. However, matrices having carboxylate anions have a negative charge only at a pH above their pKa. At a pH below the pKa, the matrices exhibit a substantially neutral charge. Suitable anionic adsorbents also include anionic adsorbents which are matrices having a combination of sulfate and carboxylate anions and phosphate anions.

Other hydrogel adsorbents which are useful for observing electrostatic or ionic charge interactions include cationic adsorbents. Specific examples of cationic adsorbents include matrices of secondary, tertiary or quaternary amines. Quaternary amines are permanently positively charged. However, secondary and tertiary amines have charges that are pH dependent. At a pH below the pKa, secondary and tertiary amines are positively charged, and at a pH above their pKa, they are negatively charged. Suitable cationic adsorbents also include cationic adsorbents which are matrices having combinations of different secondary, tertiary, and quaternary amines.

In the case of ionic interaction adsorbents (both anionic and cationic) it is often desirable to use a mixed mode ionic adsorbent containing both anions and cations. Such adsorbents provide a continuous buffering capacity as a function of pH.

Still other adsorbents which are useful for observing electrostatic interactions include dipole-dipole interaction adsorbents in which the interactions are electrostatic but no formal charge or titratable protein donor or acceptor is involved.

a) Anionic Hydrogel Adsorbent

One useful adsorbent is an anionic adsorbent such as the SAX1 ProteinChip®, made by Ciphergen Biosystems, Inc. The SAX1 protein chips are fabricated from SiO₂ coated aluminum substrates. In the process, a suspension of quaternary ammonium polystryenemicrospheres in distilled water is deposited onto the surface of the chip (1 mL/spot, two times). After air drying (room temperature, 5 minutes), the chip is rinsed with deionized water and air dried again (room temperature, 5 minutes).

b) Cationic Hydrogel Adsorbent

One useful adsorbent is an cationic adsorbent such as the SCX1 ProteinChip®, also made by Ciphergen Biosystems, Inc. The SCX1 protein chips are fabricated from SiO.sub.2 coated aluminum substrates. In the process, a suspension of sulfonate polystyrene microspheres in distilled water is deposited onto the surface of the chip (1 mL/spot, two times). After air drying (room temperature, 5 minutes), the chip is rinsed with deionized water and air dried again (room temperature, 5 minutes).

4. Coordinate Covalent Interaction Hydrogel Adsorbents

Adsorbents which are useful for observing the ability to form coordinate covalent bonds with metal ions include matrices bearing, for example, divalent and trivalent metal ions. Matrices of immobilized metal ion chelators provide immobilized synthetic organic molecules that have one or more electron donor groups which form the basis of coordinate covalent interactions with transition metal ions. The primary electron donor groups functioning as immobilized metal ion chelators include oxygen, nitrogen, and sulfur. The metal ions are bound to the immobilized metal ion chelators resulting in a metal ion complex having some number of remaining sites for interaction with electron donor groups on the analyte. Suitable metal ions include in general transition metal ions such as copper, nickel, cobalt, zinc, iron, and other metal ions such as aluminum and calcium.

a) Nickel Chelate Hydrogel Adsorbent

Another useful adsorbent is a metal chelate adsorbent such as the IMAC3 (Immobilized Metal Affinity Capture, nitrilotriacetic acid on surface) chip, also available from Ciphergen Biosystems, Inc. The chips are produced as follows: 5-Methacylamido-2-(N,N-biscarboxymethaylamino)pentanoic acid (7.5 wt %), Acryloyltri(hydroxymethyl)methylamine (7.5 wt %) and N,N′-methylenebisacrylamide (0.4 wt %) are photopolymerized, optionally using—(−)riboflavin (0.02 wt %) as a photo-initiator. Preferably, however, the use of photoinitiators is avoided as described above. The monomer solution is deposited onto a rough etched, glass coated substrate (0.4 mL, twice) and irradiated for 5 minutes with a near UV exposure system (Hg short arc lamp, 20 mW/cm2 at 365 nm). The surface is washed with a solution of sodium chloride (1 M) and then washed twice with deionized water.

The IMAC3 with Ni(II) is activated as follows. The surface is treated with a solution of NiSO₄ (50 mM, 10 mL/spot) and mixed on a high frequency mixer for 10 minutes. After removing the NiSO₄ solution, the treatment process is repeated. Finally, the surface is washed with a stream of deionized water (15 sec/chip).

5. Enzyme-Active Site Interaction Hydrogel Adsorbents

Adsorbents which are useful for observing enzyme-active site binding interactions include proteases (such as trypsin), phosphatases, kinases, and nucleases. The interaction is a sequence-specific interaction of the enzyme binding site on the analyte (typically, a biopolymer) with the catalytic binding site on the enzyme.

6. Reversible Covalent Interaction Hydrogel Adsorbents

Adsorbents which are useful for observing reversible covalent interactions include disulfide exchange interaction adsorbents. Disulfide exchange interaction adsorbents include adsorbents comprising immobilized sulfhydryl groups, e.g., mercaptoethanol or immobilized dithiothrietol. The interaction is based upon the formation of covalent disulfide bonds between the adsorbent and solvent exposed cysteine residues on the analyte. Such adsorbents bind proteins or peptides having cysteine residues and nucleic acids including bases modified to contain reduced sulfur compounds.

7. Glycoprotein Interaction Hydrogel Adsorbents

Adsorbents which are useful for observing glycoprotein interactions include glycoprotein interaction adsorbents such as adsorbents having immobilize lectins (i.e., proteins bearing oligosaccharides) therein, an example of which is CONCONAVALIN.™., which is commercially available from Pharmacia Biotech (Piscataway, N.J.). Such adsorbents function on the basis of the interaction involving molecular recognition of carbohydrate moieties on macromolecules.

8. Biospecific Interaction Hydrogel Adsorbents

Adsorbents which are useful for observing biospecific interactions are generically termed “biospecific affinity adsorbents.” Adsorption is considered biospecific if it is selective and the affinity (equilibrium dissociation constant, Kd) is at least 10.sup.-3 M to (e.g., 10.sup.-5 M, 10.sup.-7 M, 10.sup.-9 M). Examples of biospecific affinity adsorbents include any adsorbent which specifically interacts with and binds a particular biomolecule. Biospecific affinity adsorbents include for example, immobilized antibodies which bind to antigens; immobilized DNA which binds to DNA binding proteins, DNA, and RNA; immobilized substrates or inhibitors which bind to proteins and enzymes; immobilized drugs which bind to drug binding proteins; immobilized ligands which bind to receptors; immobilized receptors which bind to ligands; immobilized RNA which binds to DNA and RNA binding proteins; immobilized avidin or streptavidin which bind biotin and biotinylated molecules; immobilized phospholipid membranes and vesicles which bind lipid-binding proteins.

In a preferred embodiment, for example, the chemically selective functionality is covalently or electrostatically reactive with protein under aqueous conditions. Also, the chemically selective functionality can be, for example, an electrophilic or nucleophilic group. Also, the chemically selective functionality can be, for example, an anionic or cationic group. Also, the chemically selective functionality can be, for example, a hydrophilic or hydrophobic group. For example, the chemically selective functionality can be carboxyl, ammonium, metal chelating, or thioether. Also, the chemically selective functionality can be carboxylic acid, quaternary ammonium salt, alkylarylethyleneoxy, or ketone. Also, the chemically selective functionality can be carboxylic acid, amino, or quaternary amino group.

Monomeric subunits which include the chemically selective functionalities can be found in the following representative types of polymers and polymers: poly(2-acrylamidoglycolic acid) (WCX, Weak Cation Exchanger); poly(2-acrylamido-2-methyl-1-propanesulfonic acid) (SCX, Strong Cation Exchanger); poly(3-methacryloylamino)propyl trimethylammonium chloride (SAX, Strong Anion Exchanger); poly(2-(N,N′-biscarboxymethylamino)-6-(N-methacryloylamino)hexanoic acid) and its copolymers (IMAC, Immobilized Metal Affinity Capturer); and poly(nonylphenoxy-polyethyleneglycol methacrylate) (H50, which binds proteins through reversed phase or hydrophobic interaction chromatography). Other preferred chemically selective functionalities include 4-mercapto ethyl pyridine and mercapto benzimidazole sulfonic acid.

The copolymeric hydrogel precursor can be a copolymer comprising the first and second monomer subunits described above, and in general, the copolymer can contain but need not contain additional types of monomer subunits. Other monomeric subunits can be present in the polymeric hydrogel precursor which can be used to control the density of the binding functionalities. Selection of monomers can provide the resulting polymeric hydrogel precursor with improved water-solubility, biocompatibility, and reduced non-specific binding. Preferred monomers provide an optimal combination of such properties. Another type of monomer subunit includes the energy absorbing moieties described further below as a third monomer subunit.

The molar ratio of these first and second monomer subunits is important but not particularly limited so long as the photocrosslinkable and chemically selective functions can be achieved. For example, the amount of the first subunit must be sufficiently high to provide the copolymer with photocrosslinkability, but not so high that the crosslink density is so high that water absorption is too little to be useful. In addition, the amount of the second subunit must be sufficiently high to provide the copolymer with chemical selection capability. For example, the polymeric hydrogel precursor can be a copolymer comprising about 0.1 mole % to about 15 mol %, or more particularly, about 0.5 mol % to about 15 mol % first monomer subunit for photocrosslinkability, or more particularly about 1 mol % to about 7 mol %, or more particularly, about 0.5 mol % to about 5 mol %.

In selecting the amounts of the monomers, one skilled in the art can also consider the following. The first monomer subunit provides the copolymeric hydrogel precursor with photocrosslinkability. It provides reactive sites for both hydrogel cross-linking and surface attachment. The amount of the first subunit can be sufficiently high to provide a probe hydrogel surface with good structural features. In the SELDI applications, the probe hydrogels are used to capture analytes followed with multiple buffer washings. The hydrogels should remain integrated during these treatments in order to provide sample capturing consistency. In addition, the amount of the second monomer unit can be sufficiently high to provide the copolymer with chemical selection capability.

An important improvement of this invention is that the methods provided herein for a controlled polymerization process and cross-linking are advantageous in that they provide improved control of hydrogel precursor structure and cross-linking process, and thus, provide the hydrogels with improved consistency in the sample capturing and greater structural stability. The polymerization in bulk allows preparation of copolymeric hydrogel precursor with high molecular weight and well-defined structure. The density of the photocrosslinkable functionalities in the copolymeric hydrogel precursor can be precisely controlled, for instance, simply by changing the molar ratio of the monomer seed solution. As a result, the hydrogel does not fall apart or crack during the preparation and application as compared to the hydrogels prepared using monomer deposition methods.

Another advantageous aspect of this invention is that the pore size of the inventive hydrogels can be precisely controlled by changing the nature of and/or amount of the first monomer subunit applied. The ability to control the hydrogel porosity is important, as the pore size can be tailored to meet the specific demands of the analyte. Therefore, the hydrogels can be constructed to be capable of selectively capturing proteins and biomolecules of low molecular weight. In other embodiments the hydrogels can be constructed to be capable of binding proteins having a wide range of molecular weight.

For instance, the SAX copolymeric hydrogel precursors having 3 mol. %, 7 mol. %, and 10 mol. % of photocrosslinkable groups along the polymer backbone were prepared and cross-linked to form the corresponding hydrogel probes. It was found that a lower amount of the photocrosslinkable group provides the hydrogels with a higher binding capacity. Although the present invention is not limited by theory, these hydrogel probes are believed to have higher surface area and randomly capture more proteins and biomolecules with a broad range of molecular weight. As a result, the SELDI spectra of serum profiling are featured with an increased signal intensity and peak count, especially, in the high mass region. In contrast, the copolymeric hydrogel precursors modified with a higher amount of the photocrosslinkable group are believed to result in probe surface with a lower surface area and a smaller pore size, which favors capturing of low mass analytes. As a result, the SELDI spectra were enriched with low-mass analytes with fewer peak counts. Additional description of this is found in the working examples below. Hence, the present invention can provide the hydrogel with both the desired binding capacity and binding selectivity.

A copolymeric hydrogel precursor of the invention can be prepared by a number of routes, including free-radical polymerization and condensation polymerization, and the synthetic procedure is not particularly limited. For example, copolymerization of two types of monomers can be used to generate a copolymer with first and second types of monomer subunits.

The molecular weight of the copolymeric hydrogel precursor is not particularly limited so long as a coherent, uniform film of the precursor can be formed by, for example, solution casting or spin coating, and a solid hydrogel can be formed after photocrosslinking. In general, copolymeric hydrogel precursors with high molecular weight are preferred as they generally result in hydrogels with greater structural stability. In addition, components with low molecular weight in the copolymeric hydrogel precursor have less probability of being cross-linked and covalently fixed to the surface. It can present a potential contamination of active surface provide SELDI signal noise. Weight average molecular weight can be, for example, about 1,000 to about 10,000,000, and more particularly, about 5,000 to about 10,000,000, and more particularly, about 1,000 to about 1,000,000, and more particularly, about 5,000 to about 1,000,000, and more particularly, about 5,000 to about 500,000, and in some embodiments, about 10,000 to about 1,000,000. In general, polymeric materials are preferred, but oligomeric materials can also be used to the extent that advantages of the invention can be achieved, particularly in the SELDI and MALDI applications.

In another embodiment, a polymeric hydrogel precursor is provided comprising photocrosslinkable functionality and chemically selective functionality, wherein the precursor is prepared by functionalizing a prefunctionalized polymeric hydrogel precursor with photocrosslinkable functionality and with chemically selective functionality, wherein the amounts of photocrosslinkable functionality and chemically selective functionality provide the hydrogel precursor with the ability to be photocrosslinked into the hydrogel and the ability for the hydrogel to be selectively reactive with protein under aqueous conditions, whereby protein becomes bound to the chemically selective functionality. In this embodiment, the polymeric hydrogel precursor can be prepared by further functionalizing the functional groups of a prefunctionalized polymeric hydrogel precursor such as a hydroxyl functional polymer such as, for example, a polysaccharide. Such functional groups can be, for example, hydroxyl group, amino group, epoxy group, isocyanate group, or combinations thereof. The prefunctionalized polymeric hydrogel precursor can be, for example, based on acrylates, acrylamides, methacrylamides, vinyl polymers such as poly(vinyl alcohol), a derivative of linear, branched polysaccharides such as dextran and cellulose. Hydroxyl groups are particularly useful in the prefunctionalized polymeric hydrogel precursor. In this embodiment, for example, the polymeric hydrogel precursor can be a dextran derivative; a derivative of poly(vinyl alcohol); a derivative of poly(2-hydroxyethyl methacrylate), a derivative of poly(N-(tris(hydroxymethyl)methyl)acrylamide) or copolymer thereof. The chemically selective functionality can be introduced onto the polymers before or after the coating has been attached to the surface. For example, the hydroxyl groups of a benzophenone-modified dextran can be transformed into carboxyl groups, and the resulting dextran derivative can be then attached to the surface; or a benzophenone-modified dextran can be attached to the surface, and then hydroxyl groups in the dextran coating can be transformed into carboxyl groups.

For the prefunctionalized copolymeric hydrogel precursor, important characteristics include: (1) the polymer is water-soluble or water-swellable, facilitating fast interaction with target molecules in aqueous environments, (2) non-ionic, (3) biocompatible, (4) low non-specific adsorption, (5) reactive groups such as hydroxyl groups can be easily derivatized with a variety of functional groups, (6) high number of reactive groups provides a high capacity of binding functionality, (7) unreacted groups like hydroxyl don't interfere with protein interactions.

In a preferred embodiment, the polymeric hydrogel precursor comprises a substantially linear polymeric backbone having side groups that, respectively, comprise the photocrosslinkable functionality and the chemically selective functionality. The substantially linear polymeric backbone can be branched to the extent that the advantages of the invention can yet be achieved. Also, the linear polymeric backbone can comprise a line of cyclic units having side groups (e.g., dextran).

The photocrosslinkable hydrogel precursor composition is substantially free of photoinitiator. Rather, photoinitiation is provided by the first monomeric subunits that comprise a photocrosslinkable functionality. photoinitiators, if used at all, should not be present in amounts of more than 0.5 wt. %, and preferably amounts of more than 0.1 wt. %, and more preferably, amounts of more than 0.01 wt. %. Another advantage of the present invention is that crosslinkers and monomer diluents are not needed and are substantially absent from the photocrosslinkable hydrogel precursor composition.

Additional Embodiments: Chemically Selective Binding Functionalities and Third Monomeric Units Comprising Energy Absorbing Moieties

Additional embodiments for copolymeric hydrogel precursor and the chemically selective groups for binding in the second monomeric subunits are now described. Moreover, energy absorbing units can be present in a third type of monomeric subunit which is now described. Hence, the copolymers of the present invention can comprise (1) the first monomeric subunits in combination with the second monomeric subunits (without the third), (2) the first monomeric subunits in combination with the third monomeric subunits (without the second), and (3) the first, second, and third monomeric subunits in combination.

In some cases, the second monomeric subunits can be partially, substantially, or totally eliminated. For example, the invention also can provide a copolymeric hydrogel precursor comprising:

(a) first monomeric subunits that comprise a photocrosslinkable functionality, as described above, and

(b) third monomeric subunits that comprise one or more energy absorbing moieties, as described further below. The first and third monomeric subunits can be selected with suitable hydrophilicity and hydrophobicity to form a hydrogel and, if desired, applied in uses wherein analytes are detected such as, for example, by the mass spectral methods described herein. Optionally, the hydrogel precursor can further comprise the second monomeric subunits that comprise chemically selective functionality.

For example, the chemically selective functionalities for binding protein and other bimolecular analytes and targets in the second monomeric subunits can comprise binding functionalities which can fall into two classes which are described further below: (1) functionalities that form a covalent bond with the target, and (2) non-covalently bonding, functionalities, that form a non-covalent bond or non-covalently interact with the target.

The invention, including for these additional descriptions, encompasses both crosslinked hydrogels and hydrogel precursors, and corresponding monomers for polymer synthesis, as well as substrates coated with hydrogels and hydrogel precursors, as well as methods of making and using these hydrogels and precursors.

Chemically Selective Binding Functionalities that are Covalently Binding Moieties

Covalent bonding functional groups are useful for attaching other molecules to the hydrogel. For example, one may want to attach biomolecules, such as polypeptides, nucleic acids, carbohydrates or lipids to the hydrogel. Exemplary functional groups include:
(a) carboxyl derivatives such as N-hydroxysuccinimide esters, N-hydroxybenztriazole esters, acid halides, acyl imidazoles, thioesters, p-nitrophenyl esters, alkyl, alkenyl, alkynyl and aromatic esters;
(b) haloalkyl groups wherein the halide can be later displaced with a nucleophilic group such as, for example, a bromoacetyl group;
(c) aldehyde or ketone groups such that subsequent derivatization is possible via formation of carbonyl derivatives such as, for example, imines, hydrazones, semicarbazones or oximes, or via such mechanisms as Grignard addition or alkyllithium addition;
(d) sulfonyl halide groups for subsequent reaction with amines, for example, to form sulfonamides;
(e) reactive thiol groups, which can react with disulfides on proteins, including 2-mercaptopyridines and orthopydinyl disulfides;
(f) sulfhydryl groups, which can be, for example, acylated or alkylated;
(g) alkenes, which can undergo, for example, Michael addition, etc (e.g., maleimide);
(h) epoxides, which can react with nucleophiles, for example, amines and hydroxyl compounds;
(i) hydrazine groups, which react with sugars and glycoproteins;
(j) vinyl sulfones;
(k) activated carbonyl groups such as.

The covalent bonding functional groups can be chosen such that they do not participate in, or interfere with reactions in which they are not intended to participate in. Alternatively, the functional group can be protected from participating in the reaction by the presence of a protecting group. Those of skill in the art will understand how to protect a particular functional group from interfering with a chosen set of reaction conditions. For examples of useful protecting groups, See, Greene et al., PROTECTIVE GROUPS IN ORGANIC SYNTHESIS, John Wiley & Sons, New York, 1991.

Those of skill in the art understand that the covalent bonding functional groups discussed herein represent only a subset of functional groups that are useful in assembling the chips of the invention. Moreover, those of skill understand that the covalent bonding functional groups are also of use as components of the functionalized film and the linker arms.

As shown in Table 1, the polymer of the invention allows access to polymers having an array of covalent bonding functionalities for immobilization of binding functionalities, linker arms, binding functionality-linker arm cassettes and analytes.

TABLE 1
Protein Biochip SELECTED Reactive Chemistry

| Functional Group | Co-reactant | nucleophiles | pH |
|---|---|---|---|
| Imidazocarbonyl | NA | amine | 7-8 |
| Epoxy | NA | amine | 8-9 |
| Aldehyde | NaCNBH3 | amine | 6-9 |
| Thiol | NA | disulfide | 5.5-9 |
| Thiol | PDS | thiol | 6-8 |
| NHS | NA | amine | 6-8 |
| NHSA | NA | amine | 6-7 |
| NHM | NA | thiol | 6.5 |
| | | | 9 |
| Iodoacetyl | NA | amine | 9 |
| | | thiol | 9 |
| | | Sulfide | 9 |
| Iodoacetyl | Methionine | amine | 8.5-8.8 |
| Vinylsulfone | NA | Thiol | 7 |
| PNP | NA | amine | 8-9 |

Hepes: 4-(2-hydroxyethyl)-piperazine-1-ethanesulfonic acid

Exemplary covalent bonding functional monomers are imidazole, phenylcarboxyethanol, N-hydroxysuccinimide, N-hydroxymaleimide, cystamine/DTT, glycidol, p-nitrophenyl methylol carbonate, benzotriazoyl methylol carbonate, MeSCH₂ CH₂OH, Ellman's reagent (4-nitro-3-carboxylic acid) disulfide and O-pyridinyl-disulfide.

Chemically Selective Binding Functionalities that are Non-Covalently Binding Functionalities

Binding functionalities which do not employ covalent bonding (which also can be attached through covalent bonding functionalities) can be useful for capturing analytes from a sample for further analysis. Binding functionalities may be grouped into two classes—biospecific binding groups and chromatographic binding groups.

Non-covalent binding functionalities can be chromatographic or biospecific. Chromatographic binding functionalities bind substances via charge-charge, hydrophilic-hydrophilic, hydrophobic-hydrophobic, van der Waals interactions and combinations thereof.

Biospecific binding functionalities generally involve complementary 3-dimensional structures involving one or more of the above interactions. Examples of combinations of biospecific interactions include, but are not limited to, antigens with corresponding antibody molecules, a nucleic acid sequence with its complementary sequence, effector molecules with receptor molecules, enzymes with inhibitors, sugar chain-containing compounds with lectins, an antibody molecule with another antibody molecule specific for the former antibody, receptor molecules with corresponding antibody molecules and the like combinations. Other examples of the specific binding substances include a chemically biotin-modified antibody molecule or polynucleotide with avidin, an avidin-bound antibody molecule with biotin and the like combinations. Biospecific functionalities are generally produced by attaching the biospecific moiety through a reactive moiety, as above.

In an exemplary embodiment, the binding functionality monomer includes a binding functionality that is selected the group consisting of a positively charged moiety, a negatively charged moiety, an anion exchange moiety, a cation exchange moiety, a metal ion complexing moiety, a metal complex, a polar moiety, a hydrophobic moiety. Further exemplary binding functionalities include, an amino acid, a dye, a carbohydrate, a nucleic acid, a polypeptide, a lipid (e.g., a phosphotidyl choline), and a sugar.

Ion exchange moieties of use as binding functionalities in the polymers of the invention are, e.g., diethylaminoethyl, triethylamine, sulfonate, tetraalkylammonium salts and carboxylate.

In an exemplary embodiment, the binding functionality is a polyaminocarboxylate chelating agent such as ethylenediaminetetraacetic acid (EDTA) or diethylenetriaminepentaacetic acid (DTPA), which is attached to an amine on the substrate, or spacer arm, by utilizing the commercially available dianhydride (Aldrich Chemical Co., Milwaukee, Wis.). When complexed with a metal ion, the metal chelate binds to tagged species, such as polyhistidyl-tagged proteins, which can be used to recognize and bind target species. Alternatively, the metal ion itself, or a species complexing the metal ion can be the target.

Metal ion complexing moieties include, but are not limited to N-hydroxyethylethylenediaminoe-triacetic acid (NTA), N,N-bis(carboxymethyl)-L-lysine, iminodiacetic acid, aminohydroxamic acid, salicylaldehyde, 8-hydroxy-quinoline, N,N,N′-tris(carboxytrimethyl)ethanolamine, and N-(2-pyridylmethyl) aminoacetate. The metal ion complexing agents can complex any useful metal ion, e.g., copper, iron, nickel, cobalt, gallium and zinc.

The organic functional group can be a component of a small organic molecule with the ability to specifically recognize an analyte molecule. Exemplary small organic molecules include, but are not limited to, amino acids, heparin, biotins, avidin, streptavidin carbohydrates, glutathiones, nucleotides and nucleic acids.

In another exemplary embodiment, the binding functionality is a biomolecule, e.g., a natural or synthetic peptide, antibody, nucleic acid, saccharide, lectin, member of a receptor/ligand binding pair, antigen, cell or a combination thereof. Thus, in an exemplary embodiment, the binding functionality is an antibody raised against a target or against a species that is structurally analogous to a target. In another exemplary embodiment, the binding functionality is avidin, or a derivative thereof, which binds to a biotinylated analogue of the target. In still another exemplary embodiment, the binding functionality is a nucleic acid, which binds to single- or double-stranded nucleic acid target having a sequence complementary to that of the binding functionality.

In another exemplary embodiment, the chip of this invention is an oligonucleotide array in which the binding functionality at each addressable location in the array comprises a nucleic acid having a particular nucleotide sequence. In particular, the array can comprise oligonucleotides. For example, the oligonucleotides can be selected so as to cover the sequence of a particular gene of interest. Alternatively, the array can comprise cDNA or EST sequences useful for expression profiling.

In a further preferred embodiment, the binding functionality is selected from nucleic acid species, such as aptamers and aptazymes that recognize specific targets.

In another exemplary embodiment, the binding functionality is a drug moiety or a pharmacophore derived from a drug moiety. The drug moieties can be agents already accepted for clinical use or they can be drugs whose use is experimental, or whose activity or mechanism of action is under investigation. The drug moieties can have a proven action in a given disease state or can be only hypothesized to show desirable action in a given disease state. In a preferred embodiment, the drug moieties are compounds, which are being screened for their ability to interact with a target of choice. As such, drug moieties, which are useful in practicing the instant invention include drugs from a broad range of drug classes having a variety of pharmacological activities.

Exemplary hydrophobic adsorbent functional monomers include CH₃(CH₂)₁₇OH, 1-Octadecanol, 1-Docosanol, perfluorinated polyethyleneglycol (Sovay, USA).

Exemplary hydrophilic adsorbent functional monomers include polyvinyl alcohol) and polyvinylpyrolidone.

Exemplary anion exchange adsorbent functional monomers include 3-chloro-2-hydroxypropyl trimethylammonium chloride and 2-hydroethyl-N-methyl pyridium chloride.

Exemplary cation exchange adsorbent functional monomers include 1,4-butanediol-2-sulfonic acid, 3,5-dimethylo benzenesulfonic acid, dihydroxybenzoic acid and dimethylolacetic acid.

Exemplary metal chelate adsorbent functional monomers include N-hydroxyethylethylenediamino-triacetic acid (NTA), N,N-bis(carboxymethyl)-L-lysine, aminohydroxamic acid, salicylaldehyde, 8-hydroxy-quinoline, N,N,N′-tris(carboxytrimethyl)ethanolamine, and N-(2-pyridylmethyl) aminoacetate. The addition of a solution of metal ions, such as copper, nickel, zinc, iron and gallium functionalizes the gel.

The metal ion complexing molecules include but are not limited to those described above.

Third Monomeric Subunits Comprising EAM Functionalities

In addition, EAM (energy absorbing molecule or moiety) functionalities can be useful in the hydrogel for promoting desorption and ionization of analyte into the gas phase during laser desorption/ionization processes, and the EAM monomer, which can be a third monomer subunit different from the first and second monomer subunits, comprises a photon absorbing moiety as a functional group which can supplement the photocrosslinking group of the first monomeric subunits. The amount of the EAM can be controlled for a particular hydrogel to provide the desired analysis properties. The photon absorbing moiety preferably includes a nucleus or prosthetic group that specifically absorbs photo-radiation from a laser source. The photon absorbing groups absorbs energy from a high fluence source to generate thermal energy, and transfers the thermal energy to promote desorption and ionization of an analyte in operative contact with the hydrogel. In the case of UV laser desorption, the EAM monomer preferably includes an aryl nucleus that electronically absorbs UV photo-irradiation. In the case of IR laser desorption, the EAM monomer preferably includes an aryl nucleus or prosthetic group which preferably absorbs the IR radiation through direct vibrational resonance or in slight off-resonance fashion. A UV photon absorbing moiety can be selected from benzoic acid (e.g., 2,5 di-hydroxybenzoic acid), cinnamic acid (e.g., α-cyano-4-hydroxycinnamic acid), acetophenone, quinone, vanillic acid, caffeic acid, nicotinic acid, sinapinic acid pyridine, ferrulic acid, 3-amino-quinoline and derivatives thereof. An IR photon absorbing moiety can be selected from benzoic acid (e.g., 2,5 di-hydroxybenzoic acid), cinnamic acid (e.g., α-cyano-4-hydroxycinnamic acid), acetophenone (e.g. 2,4,6-trihyroxyacetophenone and 2,6-dihyroxyacetophenone) caffeic acid, ferrulic acid, sinapinic acid 3-amino-quinoline and derivatives thereof.

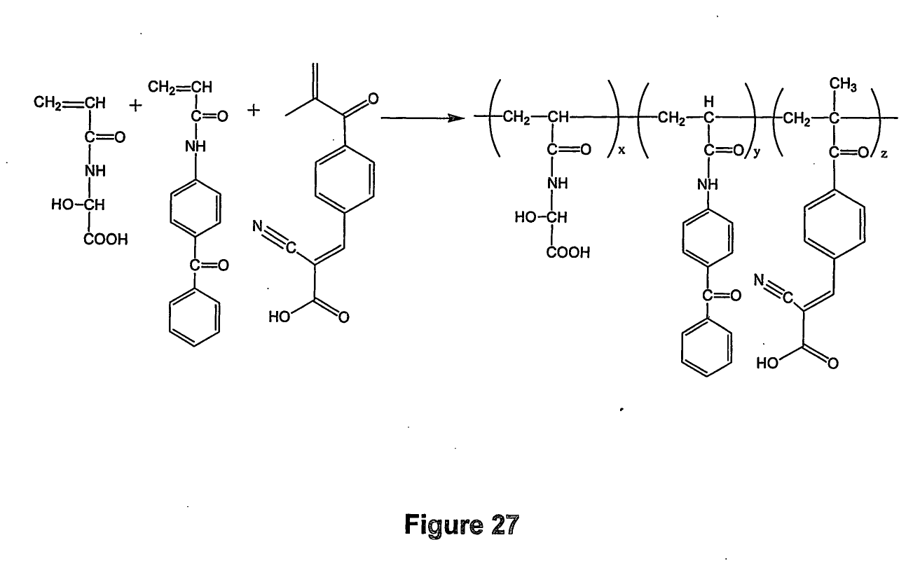

FIG. 27 illustrates an embodiment for EAM copolymeric hydrogel precursor based on three monomer units.

In one embodiment, the third monomeric subunit also comprises both an energy absorbing moiety and a chemically selective functionality for binding protein and other biomolecular analytes.

In embodiments wherein the second monomer subunits are not present in the hydrogel or its precursor, the hydrogel processing and use with a substrate and in, for example, biochip applications can be carried out as described further below.

The chips of this invention, including those comprising hydrogels comprising EAM groups, are useful for the detection of analyte molecules. When the hydrogel is functionalized with a binding group, the chip will capture onto the surface analytes that bind to the particular group. Unbound materials can be washed off. Analytes can be detected by any suitable method including a gas phase ion spectrometry method, an optical method, an electrochemical method, atomic force microscopy and a radio frequency method. Gas phase ion spectrometry methods include, e.g., mass spectrometry, ion mobility spectrometry, and total ion current measuring. Optical methods include microscopy (both confocal and non-confocal), imaging methods and non-imaging methods. Optical detection can involve detection of fluorescence, luminescence, chemiluminescence, absorbance, reflectance, transmittance, and birefringence or refractive index (e.g., surface plasmon resonance (“SPR”), ellipsometry, quartz crystal microbalance, a resonant mirror method, a grating coupler waveguide method (e.g., wavelength interrogated optical system (“WIOS”)) and interferometry). Electrochemical methods include, e.g., voltametry and amperometry techniques. Radio frequency methods include, e.g., multipolar resonance spectroscopy. Some of these methods can detect real-time binding events between an analyte and a capture molecule. Others, such as laser desorption mass spectrometry involve a surface-based analytical tool (SBAT) that requires direct physical communication with the surface of the substrate on which the analyte is captured.

II. Methodology for Functionalizing a Surface and Manufacturing a Substrate

In order to functionalize a surface with hydrogel according to the present, a four step approach can be used. In one step, a support surface is prepared, optionally with a primer layer. In another step, the polymeric hydrogel precursor is prepared in bulk. In still another step, the bulk copolymeric hydrogel precursor is physically attached to the surface substrate as a uniform polymeric coating. Finally, photocrosslinking conditions are applied such as, for example, exposure to UV light, to form the hydrogel surface coating, induce surface binding and/or fixation, and generate polymer networking.

To functionalize a surface with hydrogel, the copolymeric hydrogel precursor can be first dissolved or suspended in a single or mixed solvent system. The solvent system can be aqueous or organic. The solvent system can be partially or completely removed before use of photocrosslinking conditions. The polymeric hydrogel precursor can be then contacted with the surface to form a layer on the surface.

The method of layering the copolymeric hydrogel precursor onto the substrate surface is not particularly limited. To achieve a uniform layering, for example, methods can be used including spin coating, dip coating, roll coating, spraying, screen printing, inkjet printing, chemical vapor deposition, and other known coating methods. The coating process can be applied to an individual chip substrate. Alternatively, the individual chip substrate can be assembled into the fixture of large surface area to where the coating process can be applied. In another embodiment, the convention semiconductor process can be used: the coating solution can be applied to large flat surface to form uniform coating, and the coated wafer then can be cut into many small pieces with appropriate dimension. The diced piece can be used as a chip directly or be transferred and mounted on the chip carrier substrates. Wafer materials can be, for example, plastic, glass, silicon, metal, or metal oxide. Uniformity in the hydrogel surface coating may provide a more accurate time-of-flight analysis of samples, as all analytes absorbed on the probe surface are equidistant from an energy source of a gas phase ion spectrometer. The copolymeric hydrogel precursor, as compared to a monomer solution used for deposition and in situ polymerization and cross-linking, is of sufficient viscosity which makes the deposition hydrogel layer more compatible with established coating processes, which facilitates the formation of uniform and consistent hydrogel surfaces.

The copolymeric hydrogel precursor can be coated onto the surface either in the form of discontinuous discrete spots or continuous layers.

The photopolymerization can be regionally controlled with use of photomasks to generate patterns, as known in the art. Areas which are not exposed to light can be washed away, leaving the crosslinked hydrogel. These can be in the form of spots. For example, spots can be generated with a lateral dimension that is about 100 nm to about 3 mm, and more particularly, about 500 nm to about 500 microns.

Substrates are generally described in WO 00/66265, pages 15-17. The substrate can be made of any suitable material that is capable of supporting hydrogel material. The substrate can have various properties including porous or non-porous, rigid or flexible. The substrate surface can be in any shape including planar. However, the substrate with a flat surface would better provide the uniform polymeric coating.

Composite or multi-component substrates can be used such as, for example, two-component substrates. In a two-component substrate, for example, a solid piece of silicon or glass can be inserted in an aluminum frame-holder. A long strip can be machined out of the aluminum substrate to accommodate the glass slide or silicon wafer insert. An adhesive can be used for attachment.

Coating on aluminum substrate is a particularly preferred embodiment.

The substrate surface can be physically or chemically modified to improve adhesion of the hydrogel to the substrate. In chemical modification, for example, the substrate surface can be the surface of a primer layer that is supported by a support layer. The primer layer is not particularly limited but can be, for example, a hydrophobic primer layer. A hydrophobic primer layer is preferred, as it also can function as a passivation layer to protect the substrate surface from aqueous solution. It can be, for example, a silane primer layer, a hydrocarbon silane primer layer, a fluorinated silane primer layer, a mixed fluorinated/hydrocarbon silane primer layer, or a polymeric primer layer. When oxide substrates are used, alkoxysilane and chlorosilane chemistry can be used to form the primer layer. When noble metal substrates are used such as, for example, gold and silver, then alkanethiols or disulfides can be used to form the primer layer.

Examples of physical modification of the substrate surface include conditioning to make the surface rough, microporous, or porous.

The thickness of the primer layer is not particularly limited but can be, for example, about 4 angstroms to about 10 microns, and more particularly, about 5 nm to about 10 microns, and more particularly, about 10 nm to about 10 microns.

The type of support layer is not particularly limited but can be organic or inorganic. It can be, for example, aluminum, silicon, glass, metal oxide, metal, polymer, or composite. When the hydrogel is used as a SELDI probe, conductive supports can be used. Conducting polymers can be used. Plastic materials can be used as supports. Characteristics of plastic materials can be further changed by combining or blending different types of polymers together and by adding other materials. For instance, particulate fillers such as, for example, carbon powder, silica, ceramic, and powdered metals can be incorporated to adjust the modulus and electrical conductivity of the composite. Other additives can be used to improve chemical resistance and thermal stability.

The substrate surface, whether primed or not, can be tailored with the photocrosslinkable functionality to allow a photochemical fixation of hydrogel coating. For example, benzophenone-types of photocrosslinkable functionality can bind with C—H groups. The substrate surface can comprise photoreactive functionality to facilitate binding with the hydrogel during photocrosslinking.

The photocrosslinking can be selective such that some of the copolymeric hydrogel precursor is photocrosslinked and some of the polymeric hydrogel precursor is not. Discreet spots of crosslinked hydrogel can be formed. The remainder can be removed by, for example, washing in water. The result is a patterned surface. Traditional photolithographic methods including photomasks can be used. If the substrate surface is hydrophobic, the areas between the hydrophilic hydrogel can be hydrophobic. Hence, liquid drops of aqueous solution can be retained on a specific spot.

In a preferred embodiment, the copolymeric hydrogel precursor that is crosslinked is a substantially uniform layer on the substrate surface and has an average layer thickness of about 5 nm to about 50 microns, and more particularly, about 5 nm to about 10 microns, and more particularly, about 10 nm to about 10 microns, and more particularly about 100 nm to about 10 microns, and more particularly, about 100 nm to about 2 microns.

The thickness of these hydrogel coatings is believed to be an important aspect of the invention. Increased SELDI total signal intensity along with more SELDI peak count were observed in those embodiments which comprise a relatively thin layer having thickness under 2 μm.

The thickness of the hydrogel coatings is typically estimated with the combined measurements of reflectometry and reflectance FTIR (see working examples). The SAX hydrogel coatings with various thicknesses of ˜0.8 μm, ˜2 μm, ˜5 μm, and ˜10 μm were made. The variation of coating thicknesses was achieved by using a coating solution of 6 wt. %, 10 wt. %, 15 wt. %, and 20 wt. %, respectively, with the spin speed of ˜3,000 rpm. The polymeric hydrogel precursor used for cross-linking to obtain the hydrogels has a fixed concentration of cross-linking functionality.

Thinner hydrogel coatings having a thickness of about 0.8 μm and about 2 μm provide better SELDI signal intensity and sensitivity and higher number of the signal peak. Relatively thicker hydrogels, with a thickness of about 5 μm and about 10 μm, provide the spectra of lower SELDI intensity and lower SELDI peak count.

The present invention is not limited by theory, but a relatively thick hydrogel coating provides probe surface with more surface area and higher number of binding functional groups available for sample capturing. Hence, a higher binding capacity would be expected. However, in the process of SELDI, it is important for the bound proteins to be extracted out of the hydrogel layers and co-crystallized with EAM before being desorbed and ionized by laser excitation. It may be difficult for thick hydrogel to completely release the captured analytes in a simple extraction step, and thin hydrogel layers may thus enable the extraction and use of the captured analytes more efficiently.

Therefore, the thickness of hydrogel coatings affects not only the binding capacity, but also the extraction efficiency. The optimal thickness would be achieved by balancing of binding capacity and extraction efficiency.

The substrate can be a substrate for a biochip. In this regard, the hydrogel can be covalently bound to the substrate surface.

III. Using an Inventive Hydrogel

The copolymeric hydrogels described herein can be used for detecting analytes as described for example in the aforementioned '290 Pham publication, and WO 00/66265 to Rich et al. For example, the functionalized substrates, as described above, can be contacted with a sample that contains an analyte and then the analyte can be detected by virtue of its binding to the chemically selective functionality. Detecting the analyte can be carried out by mass spectrometric methods including use of laser desorption mass spectroscopy.

The samples are not particularly limited but can contain a biological fluid that is selected from fluids such as saliva, blood, serum, urine, lymphatic fluid, prostatic fluid, seminal fluid, milk, a cell extract and cell culture medium. In some embodiments, the sample can be pre-fractionated by size exclusion chromatography and/or ion exchange chromatography before contact with the adsorbent surface.

The sample can be contacted with the hydrogel adsorbent on the probe substrate. Then, the sample can be allowed to dry on the hydrogel adsorbent. This can result in both specific and nonspecific adsorption of the analytes in the sample by the hydrogel adsorbent, without washing away analytes that are not bound to the hydrogel adsorbent. Generally, a volume of sample containing from a few attomoles to 100 picomoles of analyte in about 1 microliter to about 500 microliters is sufficient for binding to the hydrogel adsorbent.

After the liquid sample has been removed, in certain embodiments, an energy absorbing material can be applied to the probe. Examples of energy absorbing materials include, but are not limited to, a cinnamic acid derivative, sinapinic acid, and dihydroxybenzoic acid.

After the analyte is applied to the probe and dried, it is detected using gas phase ion spectrometry. Analytes or other substances bound to the adsorbents on the probes can be analyzed using a gas phase ion spectrometer. The quantity and characteristics of the analyte can be determined using gas phase ion spectrometry. Other substances in addition to the analyte of interest can also be detected by gas phase ion spectrometry, e.g., laser desorption ionization mass spectrometry.

Gas Phase Ion Spectrometry Detection:

Data generation in mass spectrometry begins with the detection of ions by an ion detector. A typical laser desorption mass spectrometer can employ a nitrogen laser at 337.1 nm. A useful pulse width is about 4 nanoseconds. Generally, power output of about 1-25 μJ is used. Ions that strike the detector generate an electric potential that is digitized by a high speed time-array recording device that digitally captures the analog signal. Ciphergen's ProteinChip® system employs an analog-to-digital converter (ADC) to accomplish this. The ADC integrates detector output at regularly spaced time intervals into time-dependent bins. The time intervals typically are one to four nanoseconds long. Furthermore, the time-of-flight spectrum ultimately analyzed typically does not represent the signal from a single pulse of ionizing energy against a sample, but rather the sum of signals from a number of pulses. This reduces noise and increases dynamic range. This time-of-flight data is then subject to data processing. In Ciphergen's ProteinChip® software, data processing typically includes TOF-to-M/Z transformation, baseline subtraction, high frequency noise filtering.

TOF-to-M/Z transformation involves the application of an algorithm that transforms times-of-flight into mass-to-charge ratio (M/Z). In this step, the signals are converted from the time domain to the mass domain. That is, each time-of-flight is converted into mass-to-charge ratio, or M/Z. Calibration can be done internally or externally. In internal calibration, the sample analyzed contains one or more analytes of known M/Z. Signal peaks at times-of-flight representing these massed analytes are assigned the known M/Z. Based on these assigned M/Z ratios, parameters are calculated for a mathematical function that converts times-of-flight to M/Z. In external calibration, a function that converts times-of-flight to M/Z, such as one created by prior internal calibration, is applied to a time-of-flight spectrum without the use of internal calibrants.

Baseline subtraction improves data quantification by eliminating artificial, reproducible instrument offsets that perturb the spectrum. It involves calculating a spectrum baseline using an algorithm that incorporates parameters such as peak width, and then subtracting the baseline from the mass spectrum.

High frequency noise signals are eliminated by the application of a smoothing function. A typical smoothing function applies a moving average function to each time-dependent bin. In an improved version, the moving average filter is a variable width digital filter in which the bandwidth of the filter varies as a function of, e.g., peak bandwidth, generally becoming broader with increased time-of-flight. See, e.g., WO 00/70648, Nov. 23, 2000 (Gavin et al., “Variable Width Digital Filter for Time-of-flight Mass Spectrometry”).

A computer can transform the resulting spectrum into various formats for displaying. In one format, referred to as “spectrum view or retentate map,” a standard spectral view can be displayed, wherein the view depicts the quantity of analyte reaching the detector at each particular molecular weight. In another format, referred to as “peak map,” only the peak height and mass information are retained from the spectrum view, yielding a cleaner image and enabling analytes with nearly identical molecular weights to be more easily seen. In yet another format, referred to as “gel view,” each mass from the peak view can be converted into a grayscale image based on the height of each peak, resulting in an appearance similar to bands on electrophoretic gels. In yet another format, referred to as “3-D overlays,” several spectra can be overlaid to study subtle changes in relative peak heights. In yet another format, referred to as “difference map view,” two or more spectra can be compared, conveniently highlighting unique analytes and analytes which are up- or down-regulated between samples.

Analysis generally involves the identification of peaks in the spectrum that represent signal from an analyte. Peak selection can, of course, be done by eye. However, software is available as part of Ciphergen's ProteinChip® software that can automate the detection of peaks. In general, this software functions by identifying signals having a signal-to-noise ratio above a selected threshold and labeling the mass of the peak at the centroid of the peak signal. In one useful application many spectra are compared to identify identical peaks present in some selected percentage of the mass spectra. One version of this software clusters all peaks appearing in the various spectra within a defined mass range, and assigns a mass (M/Z) to all the peaks that are near the mid-point of the mass (M/Z) cluster.

Peak data from one or more spectra can be subject to further analysis by, for example, creating a spreadsheet in which each row represents a particular mass spectrum, each column represents a peak in the spectra defined by mass, and each cell includes the intensity of the peak in that particular spectrum. Various statistical or pattern recognition approaches can applied to the data.

The spectra that are generated in embodiments of the invention can be classified using a pattern recognition process that uses a classification model. In general, the spectra will represent samples from at least two different groups for which a classification algorithm is sought. For example, the groups can be pathological v. non-pathological (e.g., cancer v. non-cancer), drug responder v. drug non-responder, toxic response v. non-toxic response, progressor to disease state v. non-progressor to disease state, phenotypic condition present v. phenotypic condition absent.

In some embodiments, data derived from the spectra (e.g., mass spectra or time-of-flight spectra) that are generated using samples such as “known samples” can then be used to “train” a classification model. A “known sample” is a sample that is pre-classified. The data that are derived from the spectra and are used to form the classification model can be referred to as a “training data set”. Once trained, the classification model can recognize patterns in data derived from spectra generated using unknown samples. The classification model can then be used to classify the unknown samples into classes. This can be useful, for example, in predicting whether or not a particular biological sample is associated with a certain biological condition (e.g., diseased vs. non diseased).

The training data set that is used to form the classification model may comprise raw data or pre-processed data. In some embodiments, raw data can be obtained directly from time-of-flight spectra or mass spectra, and then may be optionally “pre-processed” as described above.

Classification models can be formed using any suitable statistical classification (or “learning”) method that attempts to segregate bodies of data into classes based on objective parameters present in the data. Classification methods may be either supervised or unsupervised. Examples of supervised and unsupervised classification processes are described in Jain, “Statistical Pattern Recognition: A Review”, IEEE Transactions on Pattern Analysis and Machine Intelligence, Vol. 22, No. 1, January 2000.

In supervised classification, training data containing examples of known categories are presented to a learning mechanism, which learns one more sets of relationships that define each of the known classes. New data may then be applied to the learning mechanism, which then classifies the new data using the learned relationships. Examples of supervised classification processes include linear regression processes (e.g., multiple linear regression (MLR), partial least squares (PLS) regression and principal components regression (PCR)), binary decision trees (e.g., recursive partitioning processes such as CART—classification and regression trees), artificial neural networks such as backpropagation networks, discriminant analyses (e.g., Bayesian classifier or Fischer analysis), logistic classifiers, and support vector classifiers (support vector machines).

A preferred supervised classification method is a recursive partitioning process. Recursive partitioning processes use recursive partitioning trees to classify spectra derived from unknown samples. Further details about recursive partitioning processes are in U.S. Pat. No. 6,675,104.

In other embodiments, the classification models that are created can be formed using unsupervised learning methods. Unsupervised classification attempts to learn classifications based on similarities in the training data set, without pre classifying the spectra from which the training data set was derived. Unsupervised learning methods include cluster analyses. A cluster analysis attempts to divide the data into “clusters” or groups that ideally should have members that are very similar to each other, and very dissimilar to members of other clusters. Similarity is then measured using some distance metric, which measures the distance between data items, and clusters together data items that are closer to each other. Clustering techniques include the MacQueen's K-means algorithm and the Kohonen's Self-Organizing Map algorithm.

The classification models can be formed on and used on any suitable digital computer. Suitable digital computers include micro, mini, or large computers using any standard or specialized operating system such as a Unix, Windows™ or Linux™ based operating system. The digital computer that is used may be physically separate from the mass spectrometer that is used to create the spectra of interest, or it may be coupled to the mass spectrometer.

The training data set and the classification models according to embodiments of the invention can be embodied by computer code that is executed or used by a digital computer. The computer code can be stored on any suitable computer readable media including optical or magnetic disks, sticks, tapes, etc., and can be written in any suitable computer programming language including C, C++, visual basic, etc.

Finally, the invention also includes particles and beads comprising the polymeric hydrogel precursors and hydrogels described above. An average diameter or size of the particles can be, for example, about 0.01 microns to about 1,000 microns, more particularly about 0.1 microns to about 100 microns, and more particularly, about 1 micron to about 10 microns. To provide consistent mass resolutions and intensities, the particles are preferably uniform in size or diameter. For example, the particles can have a coefficient of diameter variation of less than about 5%, preferably less than about 3%, more preferably less than about 1%. In one embodiment, the particles can be made of hydrogel, and the particle is substantially free of non-hydrogel material. In another embodiment, the particles can be made on non-hydrogel particles which are coated with hydrogel.

As described above, the present invention has the advantages, among others of:

(1) Excellent reproducibility, compared to on-spot grafting polymerization or in-situ grafting polymerization. The preparation of copolymers in bulk, for example, allows good control over molecular weight, uniform distribution of comonomer units, and molecular weight distribution in a very reproducible way.

(2) Copolymers synthesized in bulk can be characterized with conventional analytical tools such as, for example, NMR, GPC, and the like. These tools can be used as in processing QC. If copolymers are limited on spots, QC is difficult and limited characterization tools are available.

(3) Copolymers synthesized in bulk allow the use of a variety of separation techniques such as, for instance, size exclusion chromatography and dialysis, to completely remove low MW residues, thereby providing a hydrogel precursor being free of low molecular weight contaminants.

(4) Use of high-molecular weight polymer system as a precursor provides surface hydrogel matrix with improved cross-linking efficiency and stability

(5) The physiochemical properties of the hydrogel surface coating can be easily modulated by, for example, the concentration of the incorporated photocrosslinkable group.

(6) Covalent attachment of the copolymeric coating to the surface by photochemical reaction can produce a relatively durable surface-bound coating.

(7) The methodology of the invention allows for spatial control of crosslinking reaction, thereby empowering one to form hydrophilic/hydrophobic patterned surface. Use of agents such as, for example, hydrophobic coatings and agents from Cytonix, as described in U.S. patent publication no. 2003/0124371 A1, can be avoided in creating hydrophobic areas.

(8) The methodology of the invention also allows for independent control of polymer design, hydrogel coating thickness, hydrogel porosity, and degree of cross-linking.

(9) This inventive methodology imparts improved control over uniformity and coating thickness.

(10) The chemistry underlying the present invention can be applied to a large variety of polymers.

(11) The thin layer and porous nature of the hydrogel surface also allows unbound sample components to be readily washed out during a wash step. It can reduce non-specific bindings.

The invention is further described by reference to the following, illustrative examples, which are not limiting.

### EXAMPLE 1

### Preparation of Copolymers of Sax Monomer and Photocrosslinkable Monomer

SAX copolymers having 3 mol. %, 5 mol. %, 7 mol. %, and 10 mol. % of photocrosslinkable groups were prepared. The concentration of photocrosslinkable groups along the polymer backbone was varied in an attempt to study its effect on the stability of surface hydrogel coatings and on the protein adsorption, and provide hydrogel materials with various degrees of crosslinking.

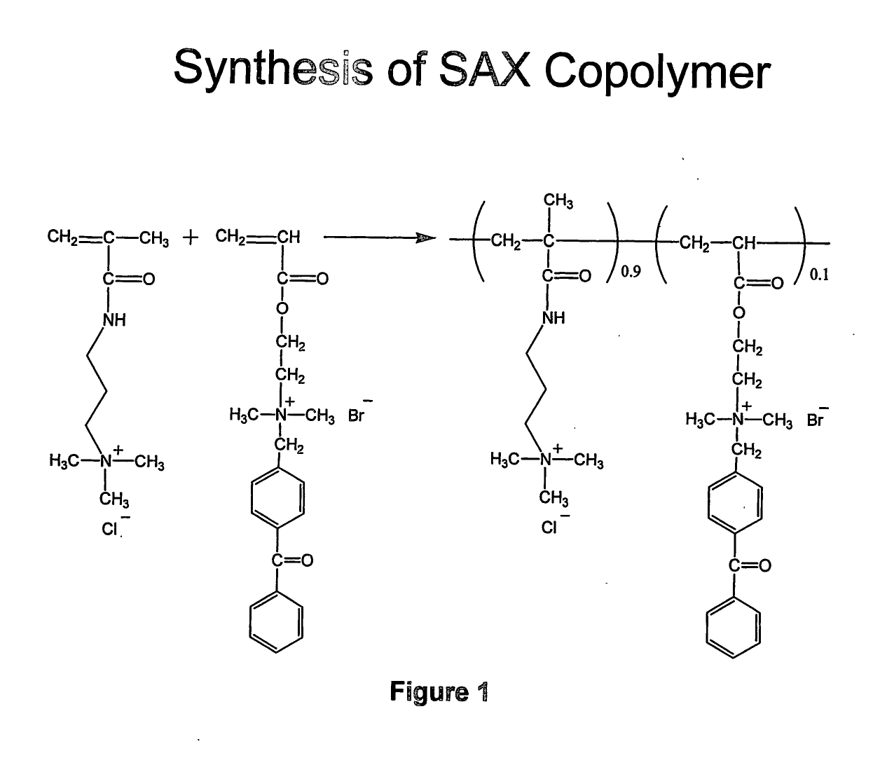

A photocrosslinkable copolymer having 10 mol. % photocrosslinkable group was prepared. More particularly, with reference to FIG. 1 below, 22 grams of 3-(methacryloylamino)propyl-trimethylammonium chloride solution (Aldrich, 50 wt. % in water) were mixed with 30 grams of distilled water, followed with 2.32 grams of 2-(acryloyloxy)ethyl](4-benzoylbenzyl) dimethylammonium bromide (Aldrich), 0.045 grams of V-50 (Wako Chemical), a water-soluble, cationic azo-initiator. The solution was purged with a flow of argon for five minutes. The vessel was sealed and then heated at 58° C. for 40 hours. The solution became very viscous after polymerization. The solution was concentrated under vacuum, and then the reaction mixture was dialyzed against DI water through a seamless cellulose tube (cutoff molecular weight, 12,000). The dialyzed polymer solution was freeze-dried under vacuum to obtain a white solid of the product. The solid powder of polymer was stored in brown vessel and used without further purification.

The same procedure was applied to prepare copolymers having 3 mol. %, 5 mol. %, and 7 mol. % photocrosslinkable groups along the polymer backbone, respectively.

### EXAMPLE 2

### Preparation of a Prime Silane Layer on Bare Aluminum Surface by Chemical Vapor Deposition (CVD) Process

Aluminum substrates were chemically cleaned with 0.01N HCl and methanol in an ultrasonic bath for 40 min, respectively. After wet cleaning, aluminum substrates were further cleaned with UV/ozone cleaner for 30 min. In the following CVD silanation process, the aluminum substrates were placed in a reaction chamber along with 3-(trimethoxysilyl) propyl methacrylate (Aldrich). A vacuum was pulled on the chamber, and the silane vaporized and reacted with the surface. The reaction was kept for 48-h for completion.

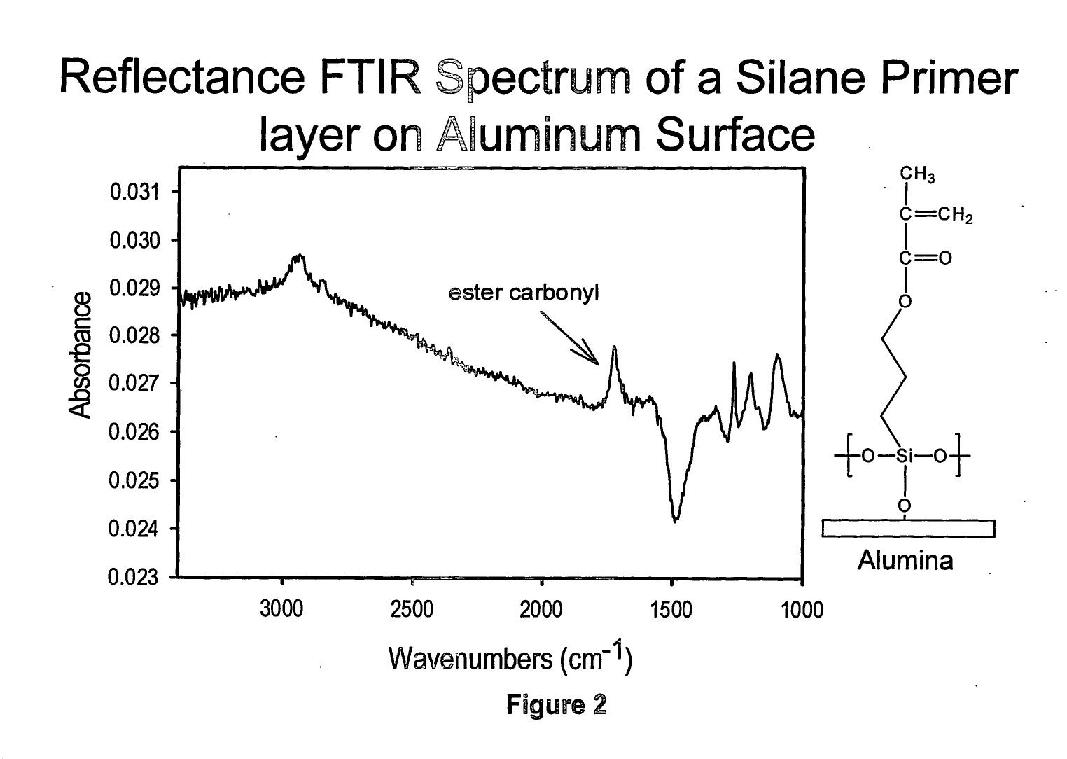

The formation of methacrylate-coated silane layers on the surface was confirmed with surface reflectance FTIR (FIG. 2) and contact angle measurements.

In another example of producing a primer silane layer by CVD process, octadecyltrichlorosilane (Aldrich) was used to replace 3-(trimethoxysilyl) propylmethacrylate to produce a hydrophobic silane layer on the surface of aluminum substrates.

### EXAMPLE 3

### Preparation of SAX Surface Hydrogel Coatings on SiO₂-Coated Aluminum Substrates

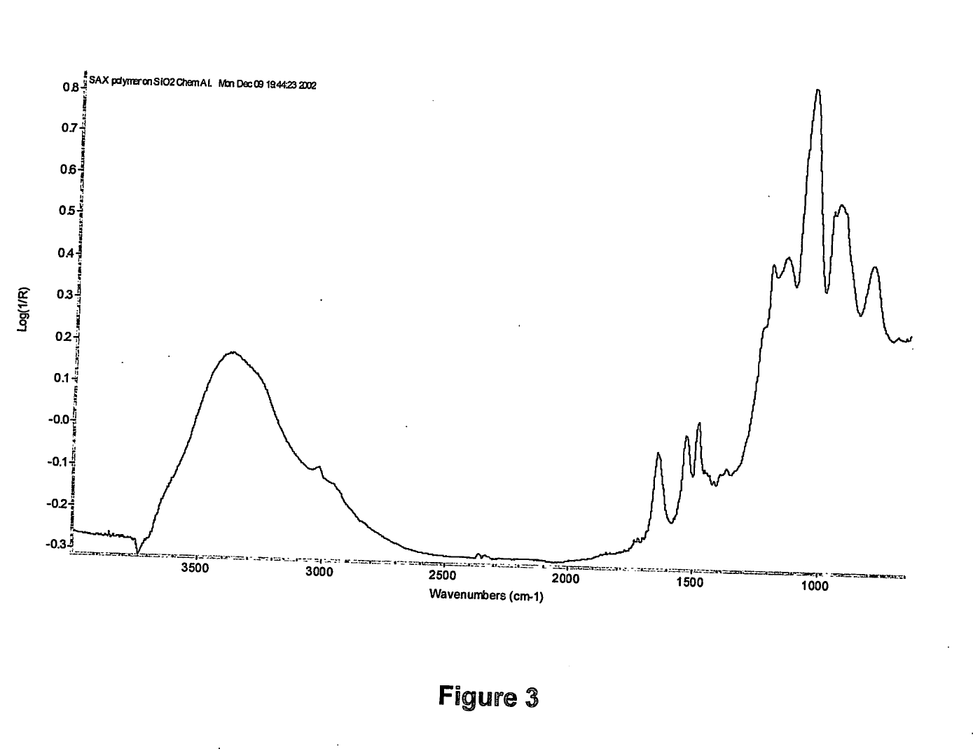

A 10 wt. % aqueous solution of SAX copolymers having 3 mol. %, 5 mol. %, 7 mol. %, and 10 mol. % of photocrosslinkable groups along the polymer backbone were dispensed on the surface of methacrylate-coated aluminum substrates, respectively. The substrates then were subjected to a process of spin-coating at 3,000 RPM for one minute. The polymer-coated chips then were exposed for 20 minutes to UV light of approximately 360 nm in wavelength (Hg short arc Lamp, 20 mW/cm² at 365 nm). Reflectance FTIR spectra (see FIG. 3) confirmed the formation of SAX hydrogel coating on the surface of aluminum substrates.

To check the stability of SAX hydrogel coatings on the surface of aluminum substrates, SAX polymeric hydrogel-coated chips were immersed in DI water for 24 h, and surface reflectance FTIR was used to follow this experiment. FTIR spectra showed, in all the cases, that there was no decrease in IR peak intensity of hydrogel coatings after 24-h water immersion. The results indicated that all the hydrogels remained on the surface after 24-h water immersion, and even an as low as 3 mol. % of photocrosslinkable group incorporated into the polymer backbone was able to fix the polymeric coating on the surface of the substrates completely.

In a control experiment, the SAX polymeric coating was prepared on non-pretreated aluminum substrates (The aluminum substrates are not subjected to the treatment of CVD silanation) and subjected to UV curing. The polymeric coating, however, didn't stay on the surface of the substrates after washing with water.

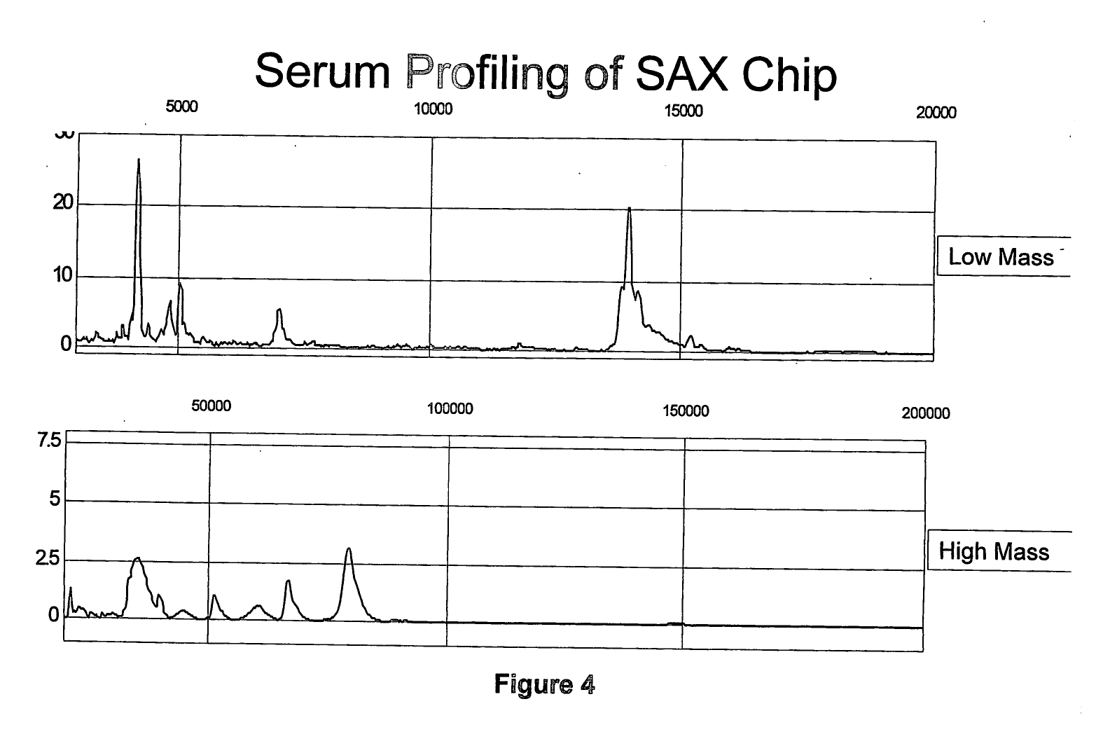

In the context of SELDI analysis, moreover, the SAX chips strongly bound albumin depleted human serum in 50 mM pH 9.0 Tris-HCl buffer solution. For protocols of using ProteinChip, see, for example, WO 00/66265 (Rich et al., “Probes for a Gas Phase Ion Spectrometer,” Nov. 9, 2000). FIG. 4 shows the composite mass spectrum at low and high molecular mass of albumin depleted human serum protein recognition profile. The profile shows the serum proteins retained on the SAX probe.

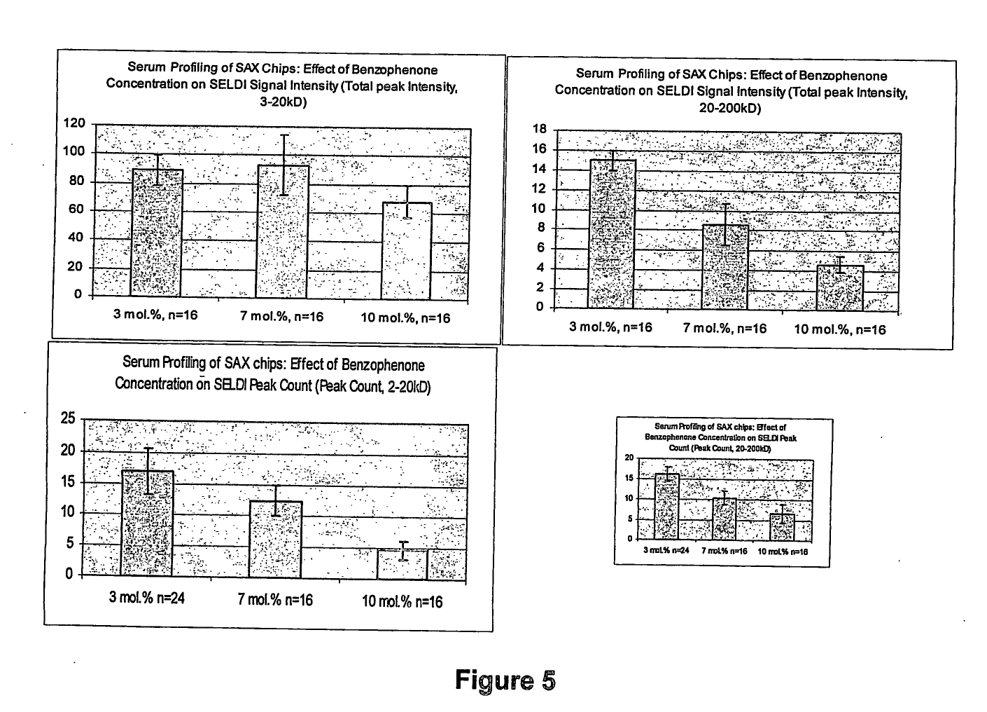

SELDI peak pattern of serum proteins recognition profile on the SAX probe is affected by concentration of the incorporated photocrosslinkable groups along the polymer backbone. In the context of the effect of hydrogel pore size on the SELDI performance, three types of SAX probe were prepared from SAX polymeric coatings with same coating thickness but different benzophenone concentration (3 mol. %, 7 mol. %, and 10 mol. % benzophenone groups along the polymer backbone, respectively). FIG. 5 shows the effect of benzophenone concentration on SELDI signal intensity and peak count of serum profiling. A lower benzophenone concentration in the polymeric composition provides a SAX probe with higher SELDI signal intensity and peak count of serum protein recognition profile and higher binding capacity, especially, at high mass region (20-200 k Dalton). In contrast, the copolymeric hydrogel precursors modified with higher amount of the photocrosslinkable group provides SAX probes with a lower surface area and a smaller pore size, which is in favor of capturing of low mass analytes. As a result, the SELDI spectra were enriched with low-mass analytes with fewer peak counts.

In the context of the effect of hydrogel coating thickness on the SELDI performance, the SAX hydrogel coatings with various thicknesses of ˜0.8 μm, ˜2 μm, 5 μm, and ˜10 μm were made and cross-linked to form the corresponding hydrogels. The thickness of the hydrogel coatings was typically estimated with the combined measurements of reflectometry and reflectance FTIR. The variation of coating thicknesses was achieved by using a coating solution of 6 wt. %, 10 wt. %, 15 wt. %, and 20 wt. %, respectively, with the spin speed of ˜3,000 rpm. The polymeric hydrogel precursor used for cross-linking to obtain the hydrogels was modified with 3 mol. % benzophenone functionality.

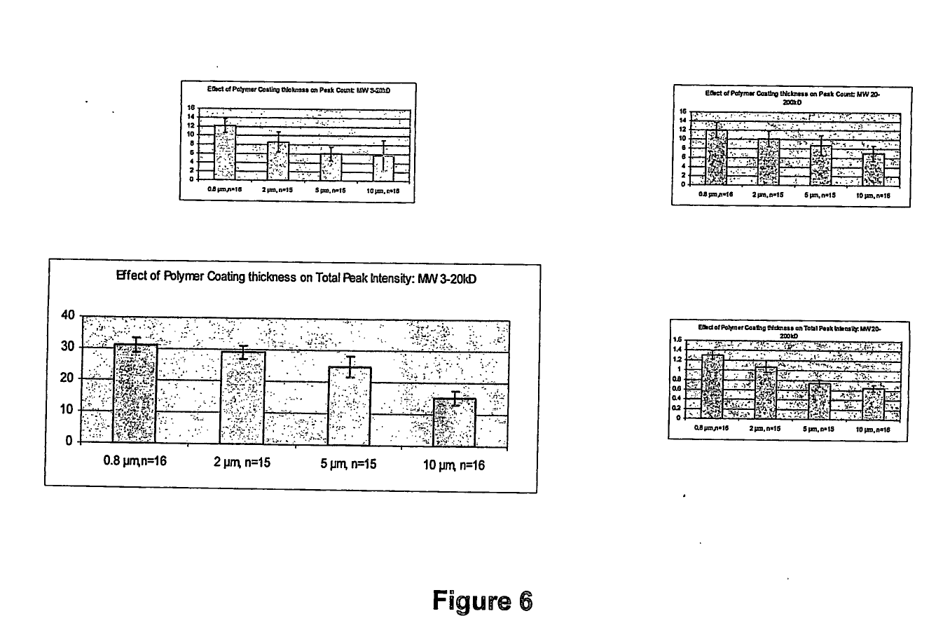

FIG. 6 shows the effect of hydrogel thickness on SELDI signal intensity and peak count of serum profiling. Thinner hydrogel coatings having a thickness of about 0.8 μm and about 2 μm provide better SELDI signal intensity and sensitivity and higher number of the signal peak. Thicker hydrogels, with a thickness of about 5 μm and about 10 μm, provide the spectra of lower SELDI intensity and lower SELDI peak count.

Increased SELDI total signal intensity along with more SELDI peak count were observed in those which comprise a thin layer having thickness under 2 μm.

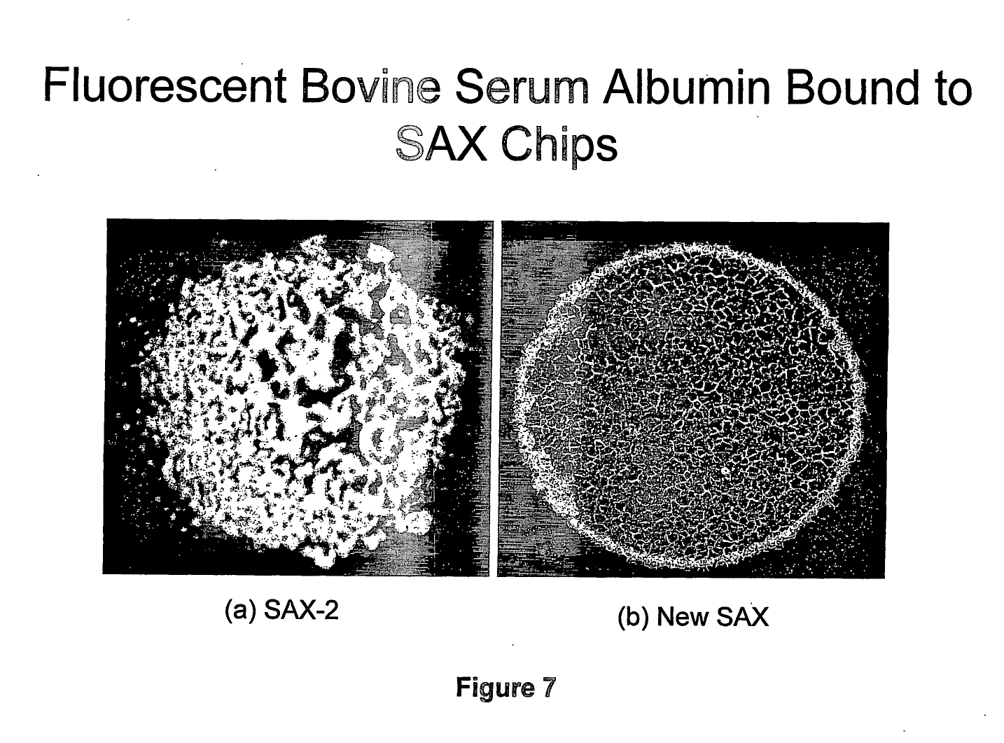

In the context of coating uniformity evaluation, the SAX chips were immersed into 50 μM bovine serum albumin tagged with 2 mol. % fluorescein in 50 mM Tris-HCl buffer solution at pH=8.3 for protein binding. After one-hour immersion, the chips were removed from the solution and rinsed with pure 50 mM Tris-HCl buffer solution and then with DI water, and dried. The bovine serum albumin bound SAX chips then were characterized with Fluorescence Microscope. A control sample of the SAX-2 probe, prepared by in-situ grafting polymerization of SAX monomer according to a reported method of PCT application WO 00/66265, was used for comparison. The fluorescent images showed that the SAX-2 chip was not fully covered with chemistry, and the hydrogel coating was not uniform. In contrast, the current inventive method provided a SAX chip with greater improvement of coating coverage and uniformity (FIG. 7).

### EXAMPLE 4

### Preparation of N-(4-benzoylphenyl)acrylamide

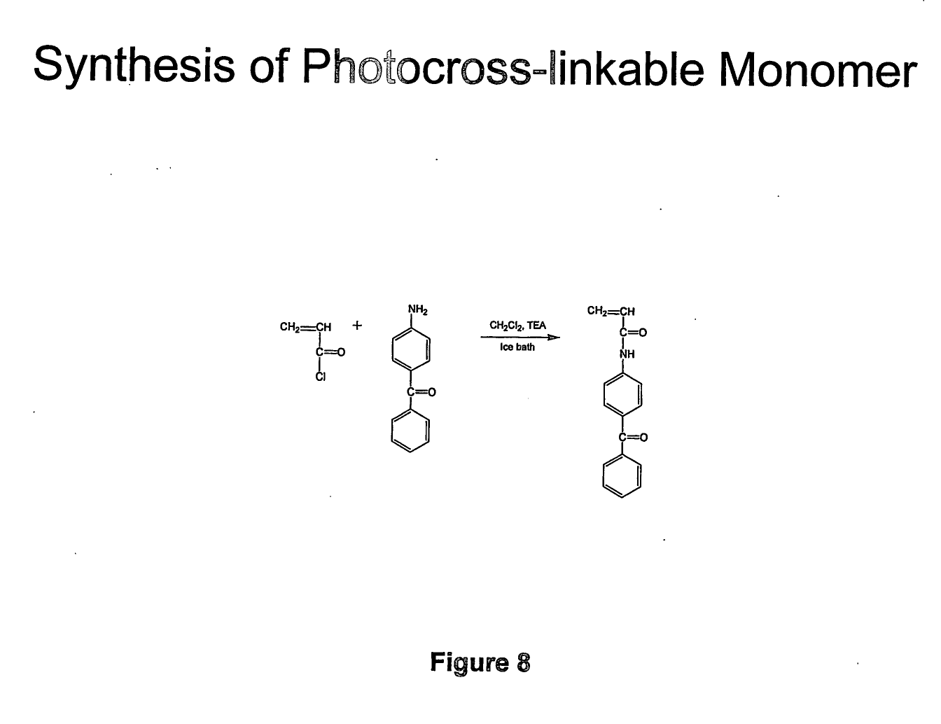

In accordance with FIG. 8, 80 mL of CH₂Cl₂ were added to a dry, 250-mL round bottom flask, equipped with a magnetic stirrer, along with 8.1 grams of 4-benzoylaniline (Aldrich), and 6.0 grams of triethylamine. The solution was cooled with an ice bath and stirred. A solution of 4.46 grams of acryloyl chloride in 20 mL of CH₂Cl₂ was added dropwise into this solution. The ice bath was then removed and the solution was warmed up to room temperature and stirred overnight. The salt precipitates are filtered off, and the CH₂Cl₂ solution is then extracted twice with 0.3 N NaOH solution, twice with 0.3 N HCl solution, and three times with DI water. The solution was dried with anhydrous MgSO₄. The CH₂Cl₂ was removed and the crude product was recrystallized from CH₂Cl₂/hexane, to give about 80% total yield of the product. ¹H NMR confirmed the formation of the desired product.

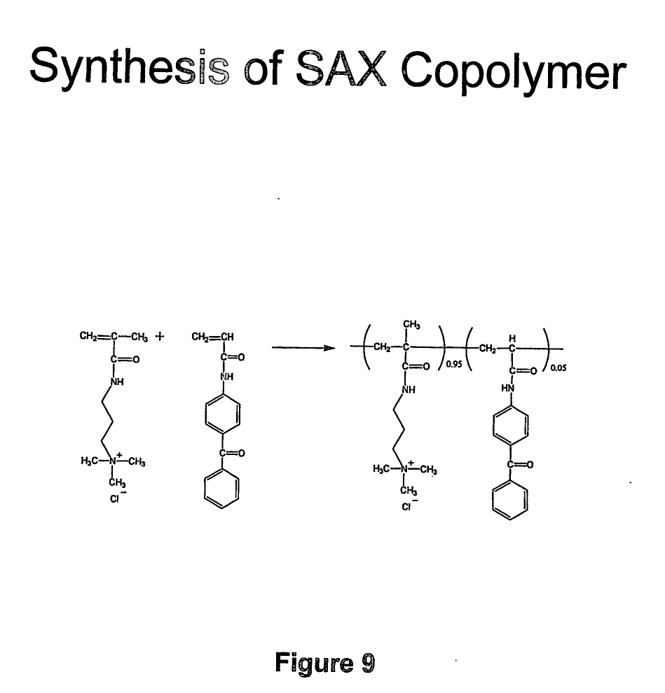

Preparation of copolymers of SAX monomer and N-(4-benzoylphenyl)acrylamide monomer: A photocrosslinkable copolymer having 5 mol. % of N-(4-benzoylphenyl)acrylamide monomer was prepared. More particularly, with reference to FIG. 9 below, 20 grams of 3-(methacryloylamino)propyl-trimethylammonium chloride solution (Aldrich, 50 wt. % in water) were mixed with 10 grams of methyl sulfoxide, and 0.045 grams of V-50 (Wako Chemical), a water-soluble, cationic azo-initiator. To this solution, 0.60 grams of N-(4-benzoylphenyl)acrylamide monomer dissolved in 10 grams of methyl sulfoxide were added dropwise. The solution was purged with a flow of argon for five minutes. The vessel was sealed and then heated at 58° C. for 40 hours. The initial solution was opaque, as N-(4-benzoylphenyl)acrylamide monomer is not completely soluble in this solvent system. However, the solution became clear and viscous as the polymerization reaction went. After polymerization, the viscous polymer solution was poured into a large excess solution of acetone to precipitate the polymers. The precipitated polymers were filtered off and re-dissoloved into DI water and the solution was dialyzed against DI water through a seamless cellulose tube (cutoff molecular weight, 12,000). The dialyzed polymer solution was freeze-dried under vacuum to obtain a white solid of the product. The solid powder of polymer was stored in brown vessel and used without further purification. This SAX copolymer has fully acrylamide structure along polymer backbone and is expected to have improved hydrolytic stability.

### EXAMPLE 5

### Preparation of Copolymer of WCX Monomer and N-(4-benzoylphenyl)acrylamide Monomer

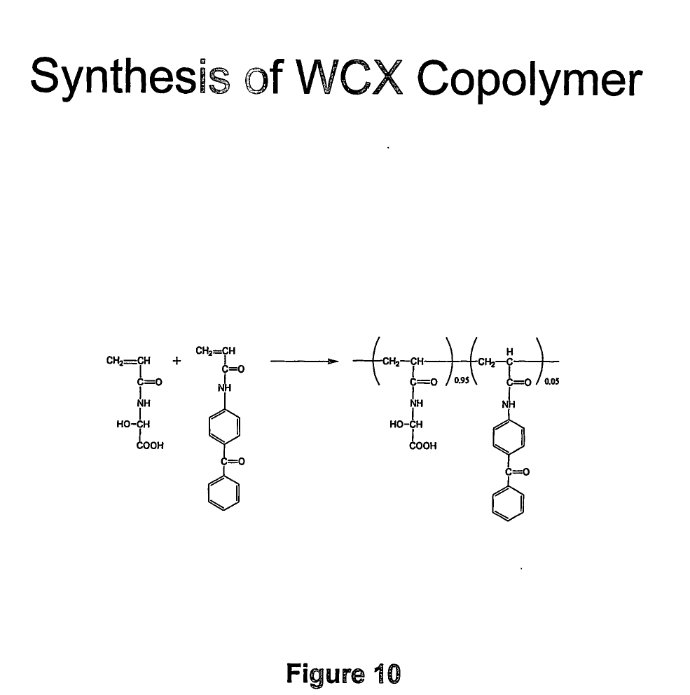

To prepare a photocrosslinkable WCX copolymer with 5 mol. % of N-(4-benzoylphenyl)acrylamide monomer along the polymer backbone (FIG. 10), 4.0 g of 2-acrylamidoglycolic acid monohydrate (WCX monomer, Aldrich) was mixed with 10.0 g of DI water and 10.0 g of DMSO, followed with 0.342 g of N-(4-benzoyl-phenyl)-acrylamide, 0.043 g of anionic azo-initiator, 4,4′-azobis-(4-cyanopentanoic acid) (Aldrich). The solution was purged with a flow of argon for five minutes. The vessel was sealed and then heated at 64° C. for 40 hours. The initial solution was opaque, but the solution became clear after polymerization. The solution was poured into a large excess solution of 2-isopropanol to precipitate the polymers. The precipitated polymers were filtered and re-dissoloved into DI water and were freeze-dried under vacuum to obtain a white solid of the product. The solid powder of polymer was stored in brown vessel.

### EXAMPLE 6

### Preparation of 4-benzoyl-N-[3-(2-methyl-acryloylamino)-propyl]-benzamide Monomer

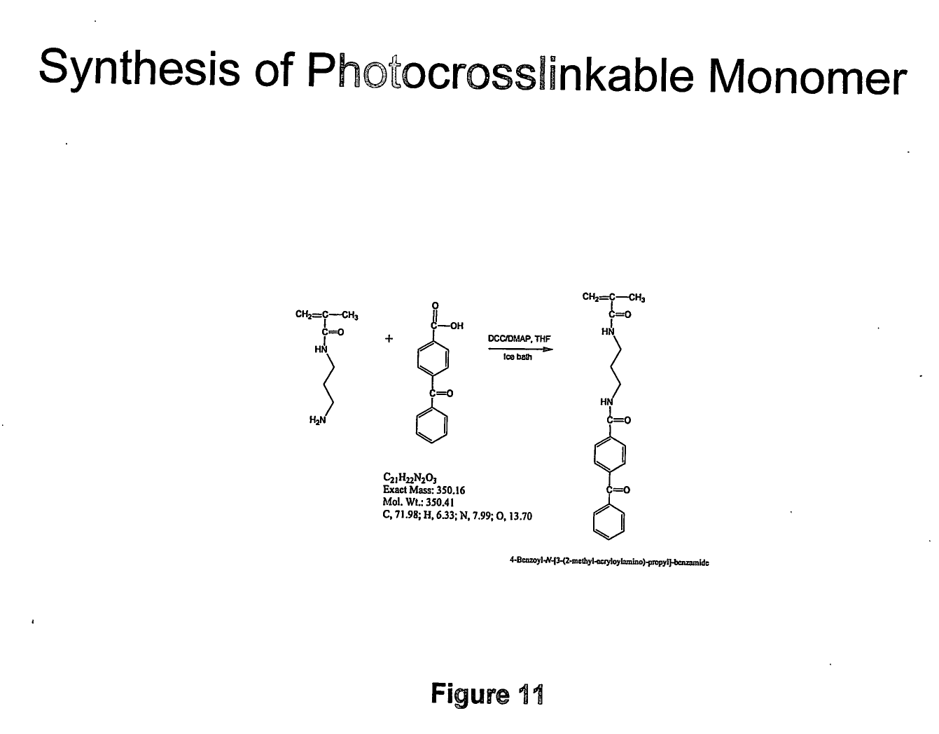

In accordance with FIG. 11, 80 mL of THF were added to a dry, 250-mL round bottom flask, equipped with a magnetic stirrer, along with 4.82 grams of N-(3-aminopropyl)methacrylamide hydrochloride (Polysciences, Warrington, Pa. 18976), 6.10 grams of 4-benzoylbenzoic acid (Aldrich), 5.60 grams of 1,3-dicyclohexylcarbodiimide (DCC), 0.4 gram of dimethyaminopyridine, and 5.5 grams of triethylamine. The solution was cooled with an ice bath and stirred for 3 hours. The ice bath was removed and the solution was stirred at room temperature overnight. After then, the precipitates were filtered off and the solvent was evaporated. The residual was re-dissolved in CHCl₃. The solution was extracted twice with 0.3 N NaOH solution, twice with 0.3 N HCl solution, and three times with DI water. The chloroform was removed and the crude product was recrystallized from chloroform/toluene, to give about 60% total yield of the product. ¹H NMR confirmed the formation of the desired product.

### EXAMPLE 7

### Preparation of Copolymer of WCX Monomer and 4-benzoyl-N-[3-(2-methyl-acryloylamino)-propyl]-benzamide Monomer

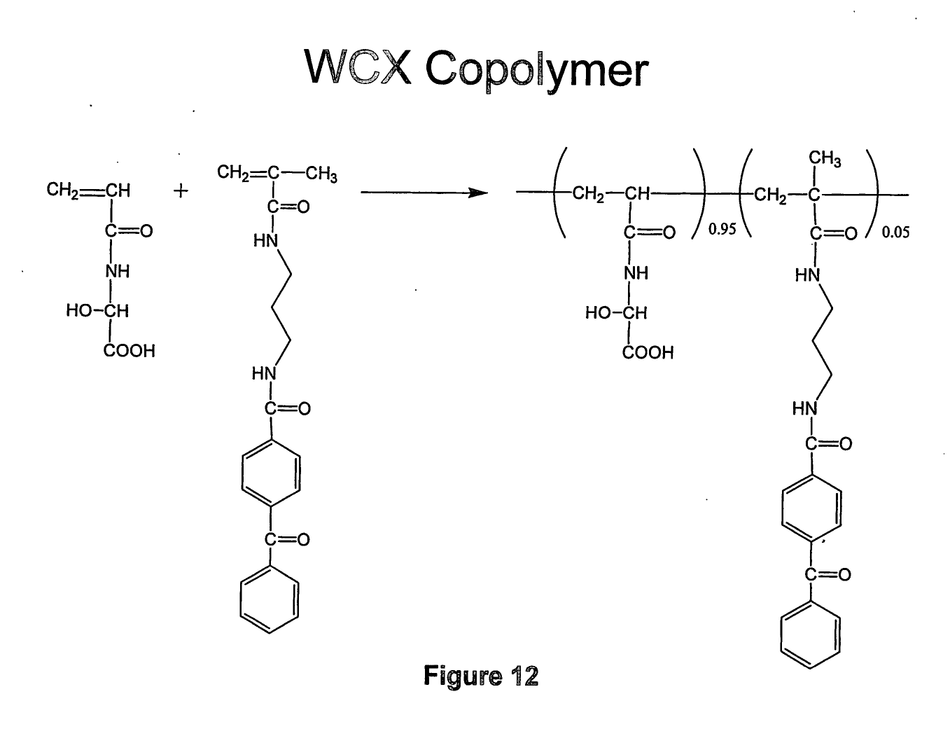

To prepare a photocrosslinkable WCX copolymer having 5 mol. % benzophenone along the polymer backbone (FIG. 12), 8.0 g of 2-acrylamidoglycolic acid monohydrate (WCX monomer, Aldrich) was mixed with 20.0 g of DI water and 20.0 g of DMSO, followed with 0.9038 g of 4-benzoyl-N-[3-(2-methyl-acryloylamino)-propyl]-benzamide, 0.126 g of anionic azo-initiator, 4,4′-azobis-(4-cyanopentanoic acid) (Aldrich). The solution was purged with a flow of argon for five minutes. The vessel was sealed and then heated at 64° C. for 48 hours. The solution became viscous after polymerization, indicating the formation of copolymers with high molecular weight.

### EXAMPLE 8

### Preparation of WCX Surface Hydrogel Coatings

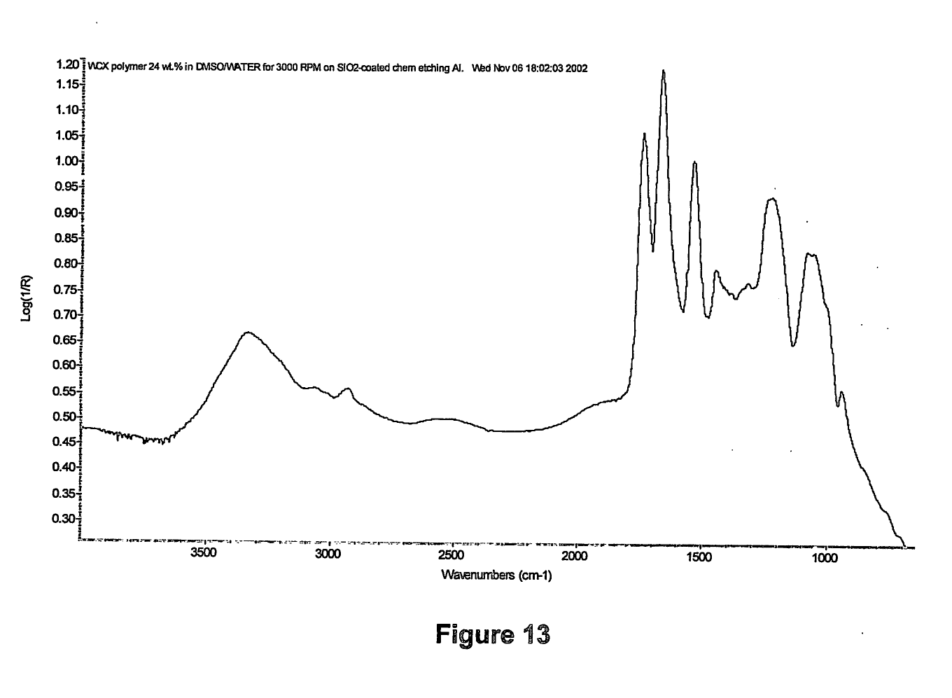

A 24 wt. % solution of WCX copolymer in water/DMSO (1:1, w/w) was used to prepare hydrogel coatings on methacrylate silaned SiO₂-coated aluminum chips. The solution was dispensed on the surface of aluminum substrates and subjected to a process of spin-coating at 3,000 RPM for one minute. The WCX polymer-coated chips were exposed to UV irradiation (Hg short arc Lamp, 20 mW/cm² at 365 nm) for 20 minutes. Reflectance FTIR spectrum indicated the formation of WCX hydrogel on the chip, and the coating was stable after water washing and buffer washing (FIG. 13).

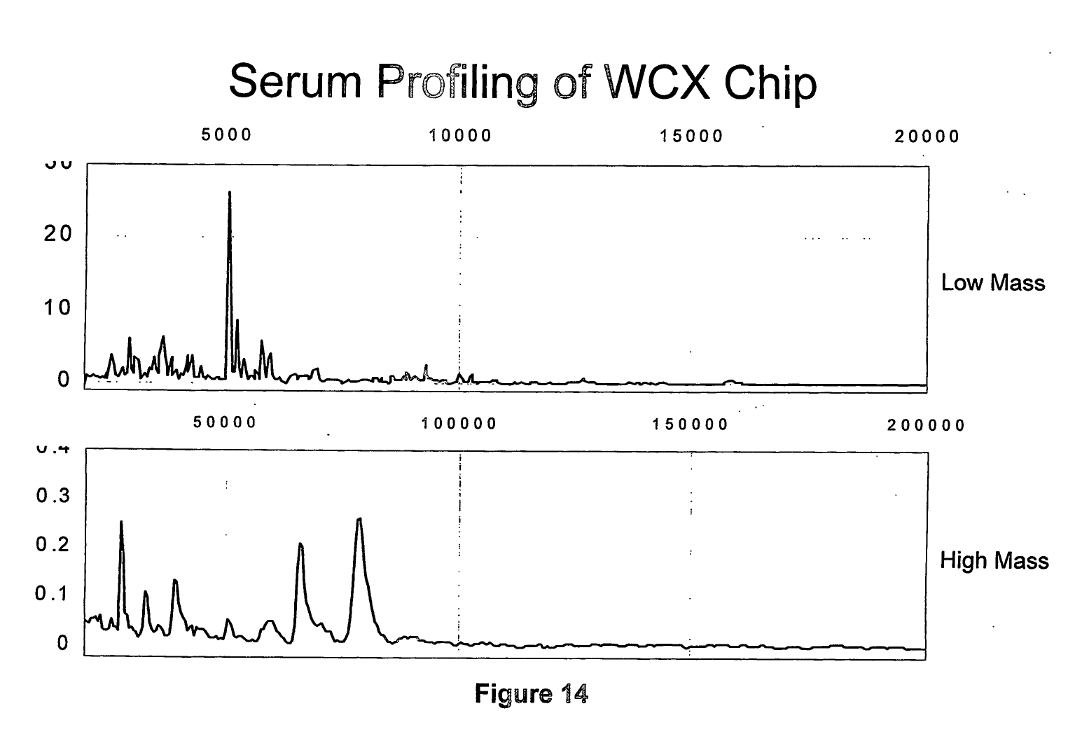

In the context of SELDI analysis, moreover, the WCX chips strongly bound albumin depleted human serum proteins in 50 mM pH 5.0 sodium acetate buffer solution. For protocols of using ProteinChip, see, for example, WO 00/66265 (Rich et al., “Probes for a Gas Phase Ion Spectrometer,” Nov. 9, 2000). FIG. 14 shows the composite mass spectrum at low and high molecular mass of albumin depleted human serum proteins recognition profile. The profile shows the serum proteins retained on the WCX probe.

### EXAMPLE 9

### Preparation of H50 Copolymer of Nonylphenoxy-Polyethyleneglycol Methacrylate Monomer and 4-benzoyl-N-[3-(2-methyl-acryloylamino)-propyl]-benzamide Monomer

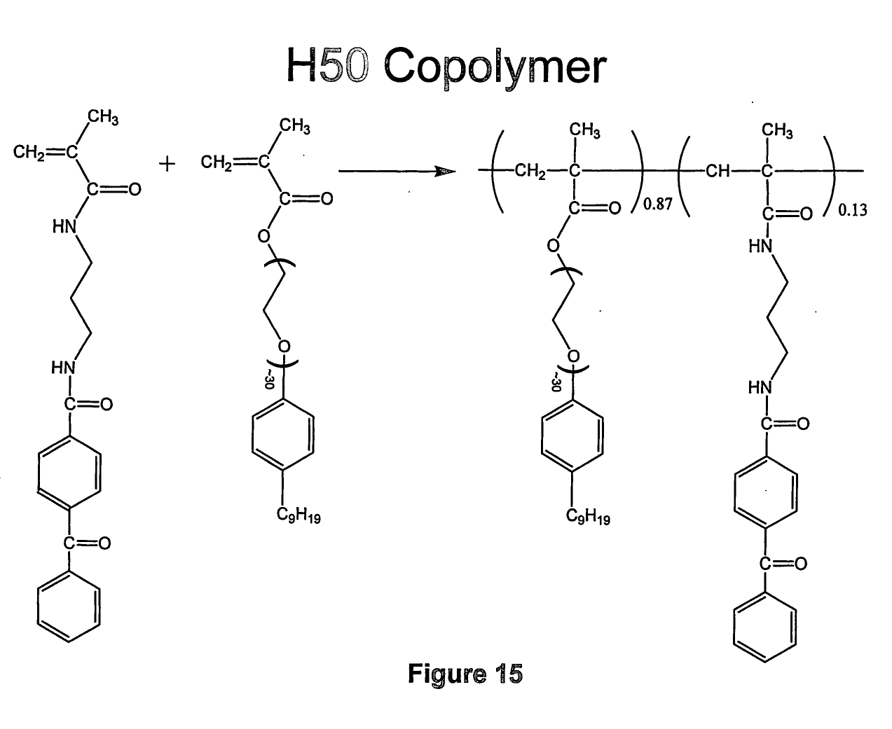

The procedure for the preparation of poly(nonylphenoxy-polyethyleneglycol methacrylate) (H50, which binds proteins through reversed phase or hydrophobic interaction chromatography) photocrosslinkable copolymer with 13 mol. % of benzophenone along the polymer backbone was as follows (FIG. 15): 0.2160 g of nonylphenoxy-polyethyleneglycol methacrylate monomer was mixed with 0.007 g of 4-benzoyl-N-[3-(2-methyl-acryloylamino)-propyl]-benzamide and 0.006 g of azo-initiator, 2,2′-azobisisobutyronitrile (AIBN, Aldrich) and 3 mL of dioxane. The solution was purged with a flow of argon for five minutes. The vessel was sealed and then heated at 62° C. for 48 hours. The solution became viscous after polymerization, indicating the formation of copolymers with high molecular weight.

### EXAMPLE 10

### Preparation of H50 Surface Hydrogel Coatings

A ˜10 wt. % solution of H50 copolymer in dioxane was used to prepare hydrogel coatings on methacrylate silaned SiO₂-coated aluminum chips. The solution was dispensed on the surface of aluminum substrates and subjected to a process of spin-coating at 3,000 RPM for one minute. The H50 copolymer-coated chips were dried and then exposed to UV irradiation (Hg short arc Lamp, 20 mW/cm² at 365 μm) for 20 minutes. Reflectance FTIR results indicated the formation of H50 hydrogel on the chip surface, and the coatings withstand with buffer washing.

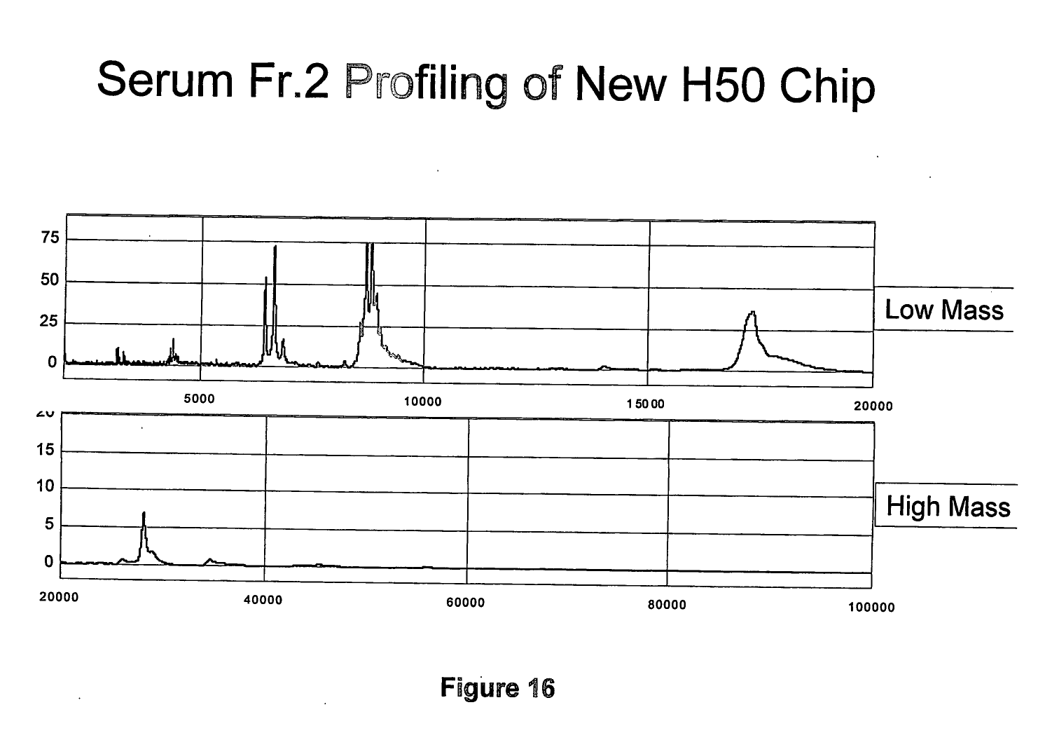

In the context of SELDI analysis, moreover, the H50 chips strongly bound fractionated albumin depleted human serum proteins in 0.5 wt. % trifluoroacetic acid buffer solution. For protocols of using ProteinChip, see, for example, U.S. patent publication No. 2003/0124371 A1. FIG. 16 shows the composite mass spectrum at low and high molecular mass of albumin depleted human serum protein fraction 2 recognition profile. The profile shows the serum proteins retained on the H50 probe.

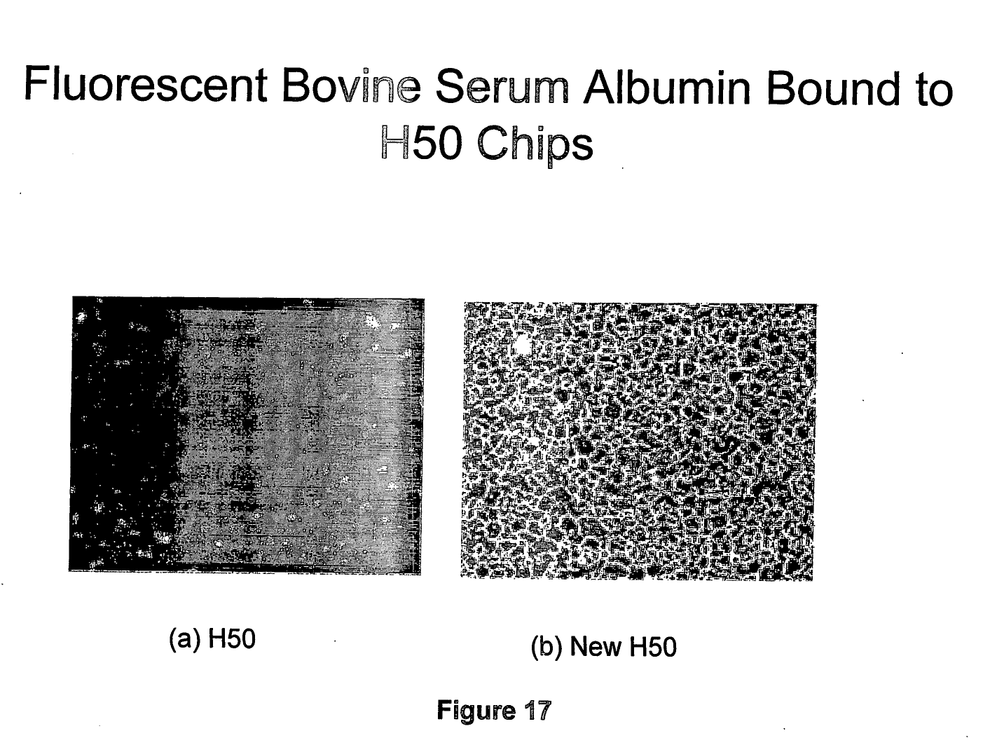

In the context of coating uniformity evaluation, the new H50 chips prepared using the inventive method were immersed into 50 μM bovine serum albumin tagged with 2 mol. % fluorescein in 0.5 wt. % trifluoroacetic acid buffer solution for capturing. After immersion, the chips were removed from the solution and rinsed with pure 0.5 wt. % trifluoroacetic acid buffer solution and then with DI water, and dried. The bovine serum albumin bound H50 chips then were characterized with Fluorescence Microscope. A control sample of the H50 probe, prepared by in-situ grafting polymerization of H50 monomer according to a reported method of U.S. patent publication No. 2003/0124371 A1, was used for comparison. The fluorescent images showed that the H50 control chip was not fully covered with chemistry, and the hydrogel coating was not uniform. In contrast, the current inventive method provided a H50 chip with greater improvement of coating coverage and uniformity (FIG. 17).

### EXAMPLE 11

### Preparation of IMAC Copolymer of 5-methacylamido-2-(N,N-biscarboxymethaylamino)pentanoic Acid Monomer and acryloyltri(hydroxymethyl)methylamine and 4-benzoyl-N-[3-(2-methyl-acryloylamino)-propyl]-benzamide Monomer

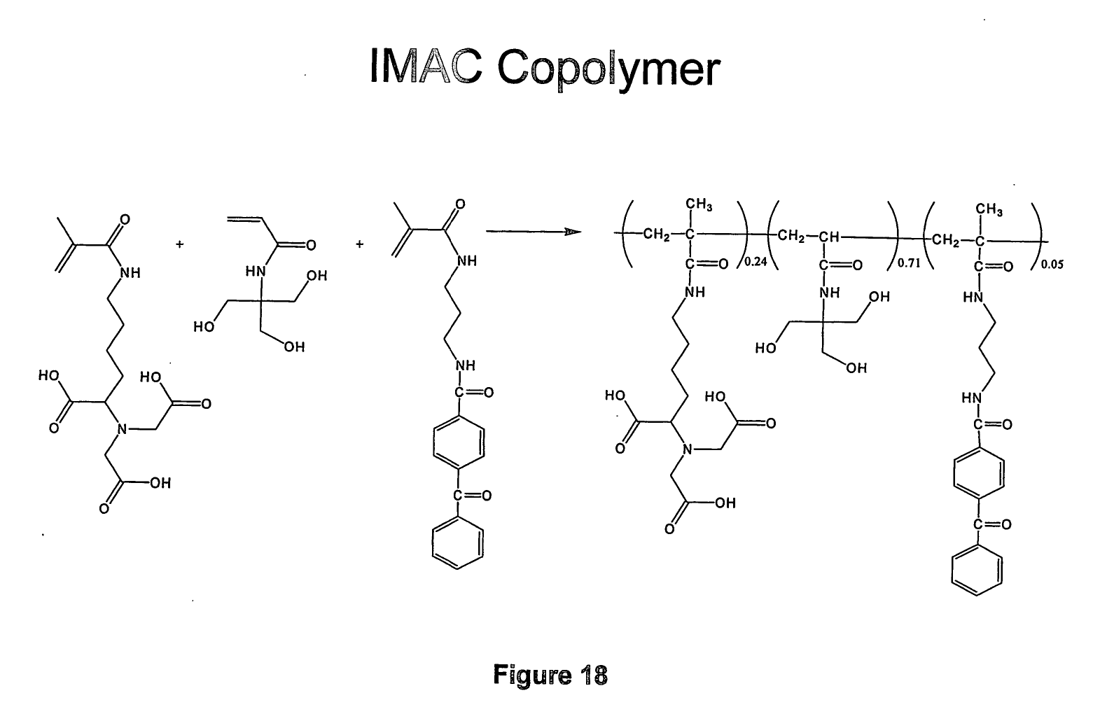

To prepare a photocrosslinkable IMAC copolymer having 5 mol. % benzophenone along the polymer backbone, with reference to FIG. 18 below, 1.68 grams of 5-methacylamido-2-(N,N-biscarboxymethaylamino)pentanoic acid monomer dissolved in 20 grams of DI water were mixed with 2.67 grams of acryloyltri-(hydroxymethyl)methylamine and 0.054 grams of 4,4-azobis(4-cyanovaleric acid) (Aldrich), a water-soluble, anionic azo-initiator, and 5 grams of methyl sulfoxide. To this solution, 0.375 grams of 4-benzoyl-N-[3-(2-methyl-acryloylamino)-propyl]-benzamide dissolved in 5 grams of methyl sulfoxide were added dropwise. The solution was purged with a flow of argon for five minutes. The vessel was sealed and then heated at 63° C. for 40 hours. The solution became very viscous after polymerization. The polymer solution was poured into a large excess solution of 2-isopropanol to precipitate the polymers. The precipitated polymers were filtered off and re-dissoloved into DI water and freeze-dried under vacuum to obtain the product.

### EXAMPLE 12

### Preparation of IMAC Surface Hydrogel Coatings

A ˜20 wt. % solution of IMAC copolymer dissolved in water under basic condition was used to prepare hydrogel coatings on methacrylate silaned SiO₂-coated aluminum chips. The solution was dispensed on the surface of aluminum substrates and subjected to a process of spin-coating at 3,000 RPM for one minute. The IMAC polymer-coated chips were dried and then exposed to UV irradiation (Hg short arc Lamp, 20 mW/cm² at 365 nm) for 20 minutes. Reflectance FTIR study indicated the formation of IMAC hydrogel on the chip surface, and the coatings withstand with buffer washes.

### EXAMPLE 7

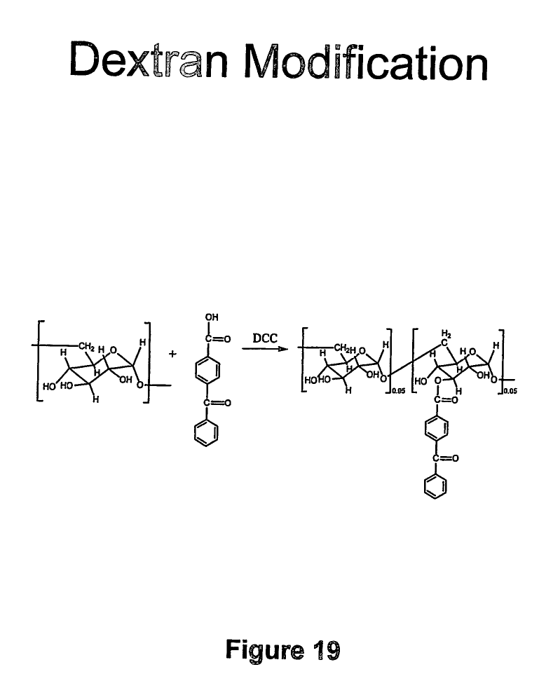

Preparation of surface hydrogel coatings based on dextran chemistry Dextran was coupled with 4-benzoylbenzoic acid to prepare benzophenone-modified dextran (FIG. 19). The synthetic procedure is as follows:

30 mL of DMSO were added to a dry, 250-mL round bottom flask, equipped with a magnetic stirrer, along with 7.07 of dextran (Mw 69,000, Sigma), 0.99 grams of 4-benzoylbenzoic acid, 1.78 grams of 1,3-dicyclohexylcarbodiimide (DCC), 0.4 gram of dimethyaminopyridine, and 2.0 grams of triethylamine. The solution was cooled with an ice bath and stirred for 3 hours. The ice bath was removed and the solution was stirred at room temperature overnight. After then, the precipitated DBU by-product was filtered off, the filtrate was poured into acetone to precipitate the polymer. The polymer precipitates were re-dissolved in DI water, and the solution mixture was dialyzed against DI water through a seamless cellulose tube (cutoff molecular weight, 12,000). The dialyzed polymer solution was freeze-dried under vacuum, yielding a white solid of the product.

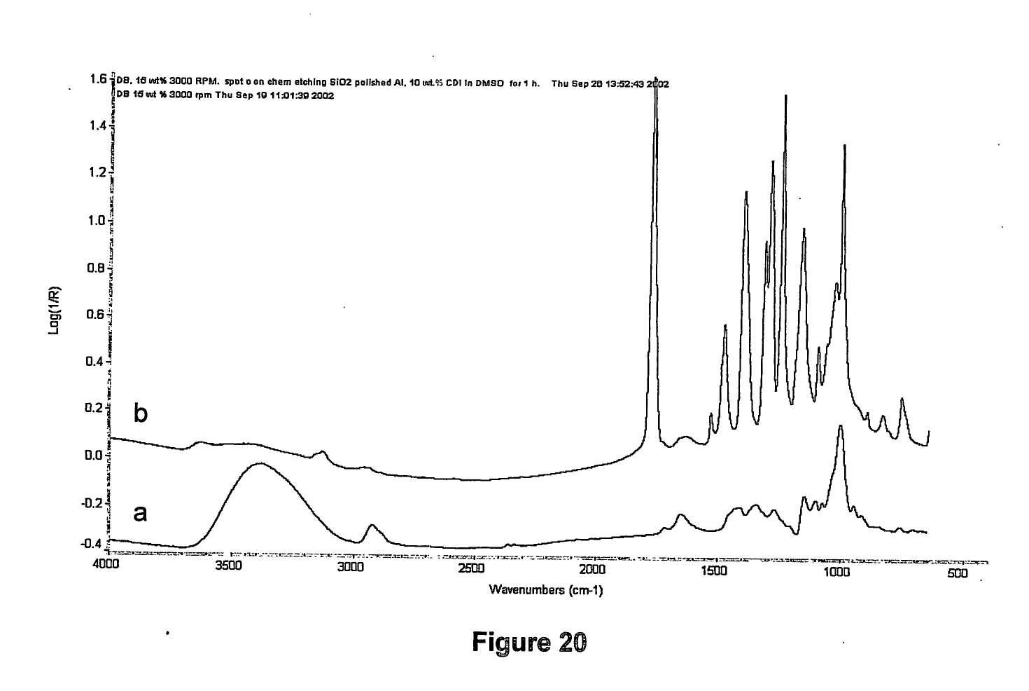

A 15 wt. % solution of benzophenone-modified dextran in DI water/ethanol (4/1, v/v) was prepared and dispensed on the surface of methacrylate-coated aluminum substrates, the substrates were subjected to a process of spin-coating at 3,000 RPM for one minute. The polymer-coated chips then were exposed for 20 minutes to UV light of approximately 360 nm in wavelength (Hg short arc Lamp, 20 mW/cm² at 365 nm). Reflectance FTIR spectrum (see FIG. 20 (a)) confirmed the formation of dextran hydrogel coating on the surface of aluminum substrates. The obtained coating was stable against water washing for 24 h.

The dextran-coated chip was reacted with 1,1′-carbonyldiimidazole (CDI, Aldrich) to prepare pre-activated surface (PS). The synthetic procedure was as follows:

The dextran-coated chips were immersed into a 10 wt. % solution of carbonyldiimidazole (CDI) in DMSO for one hour. The chips then were removed from the solution and washed with DMSO followed with acetone, dried with a flow of nitrogen. Reflectance FTIR spectrum (see FIG. 20 (b)) confirmed nearly quantitative conversion of the hydroxyl groups to the imidazole carboxylic ester, as indicated by the nearly complete disappearance of the hydroxyl peak (3500 to 3300 cm⁻¹) and the formation of a strong carbonyl peak at 1771 cm⁻¹.

These CDI-activated chips are designed specifically for immunoassay, receptor-ligand binding and DNA-binding protein applications.

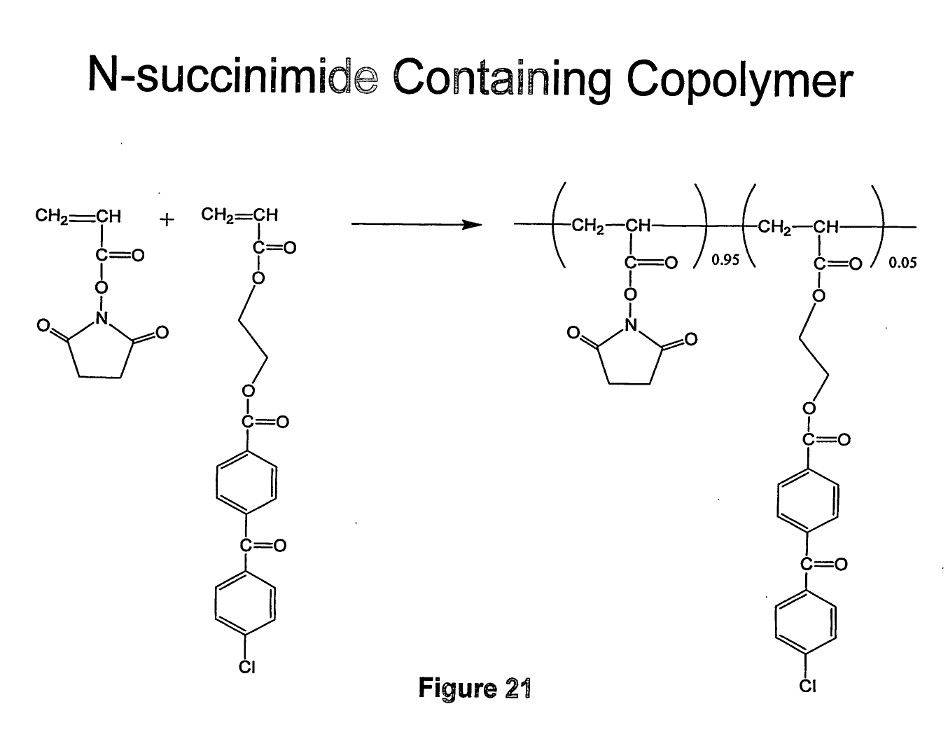

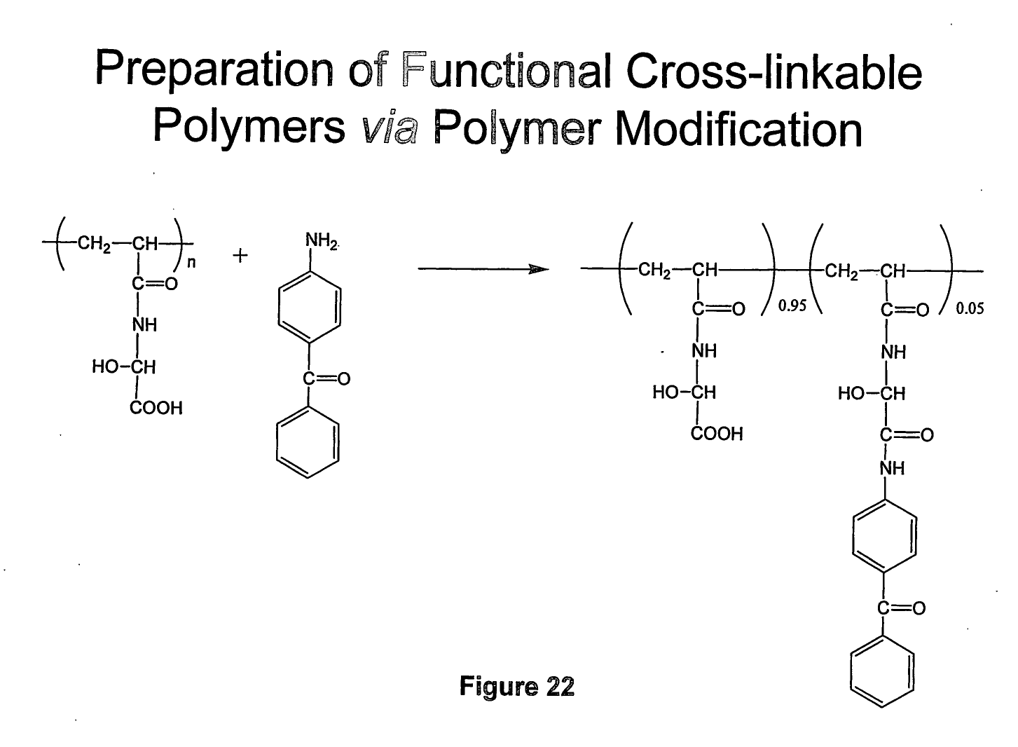

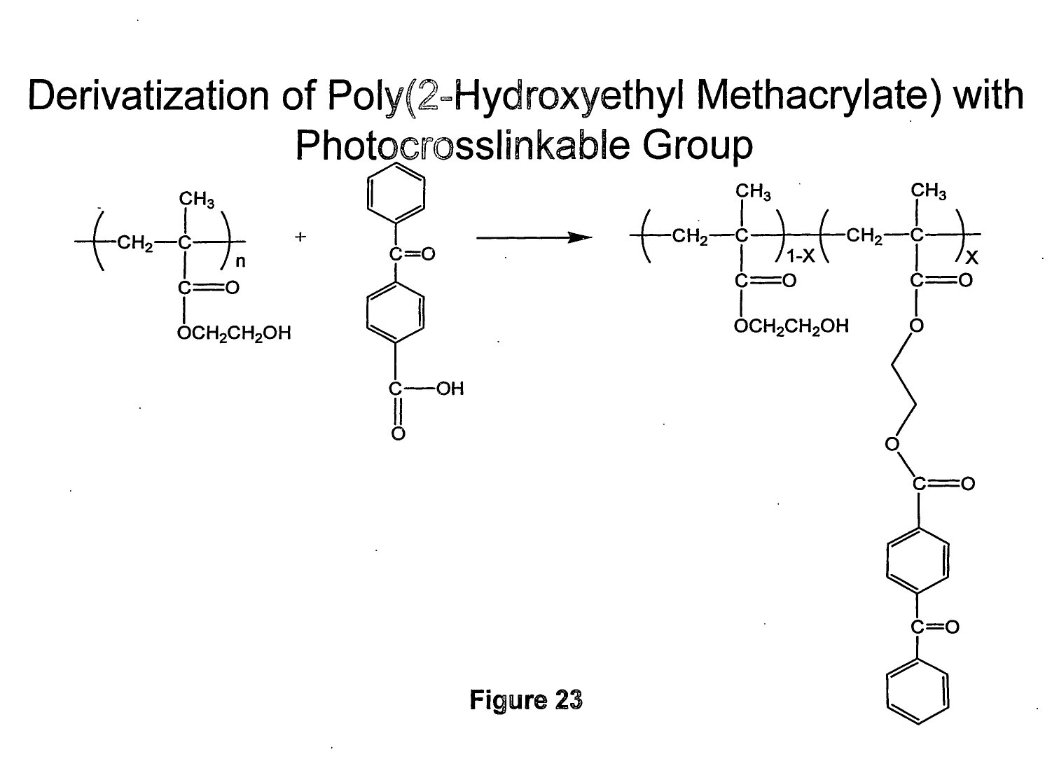

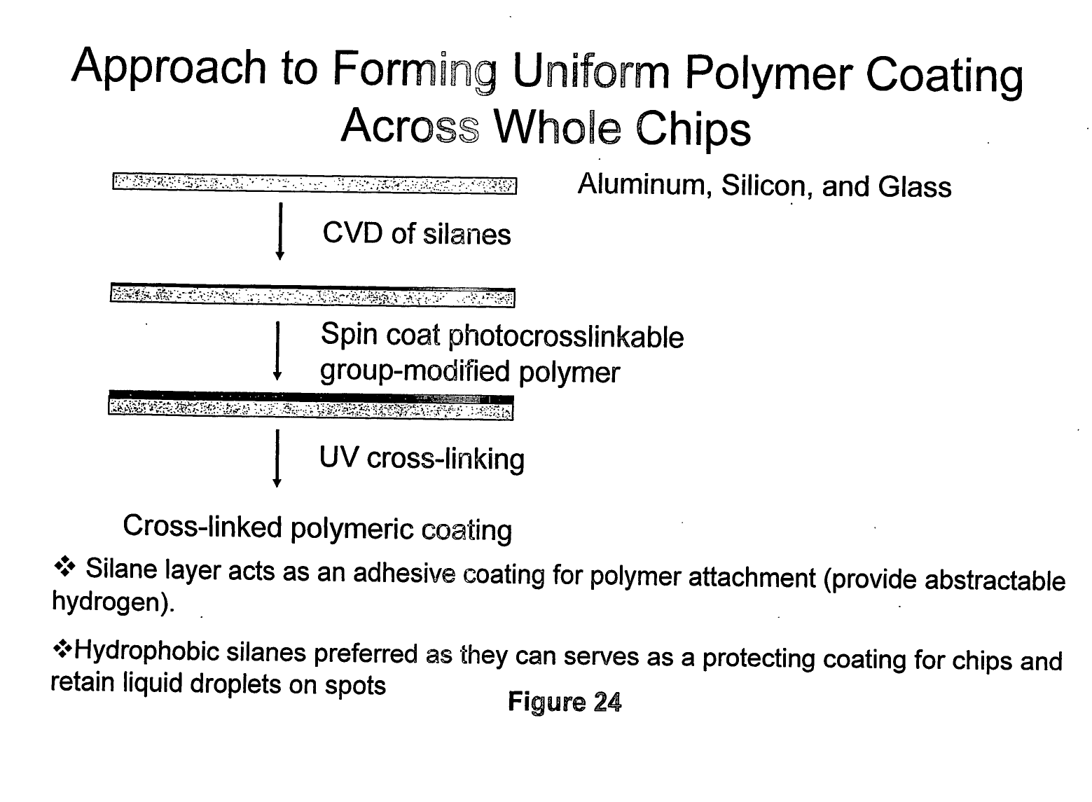

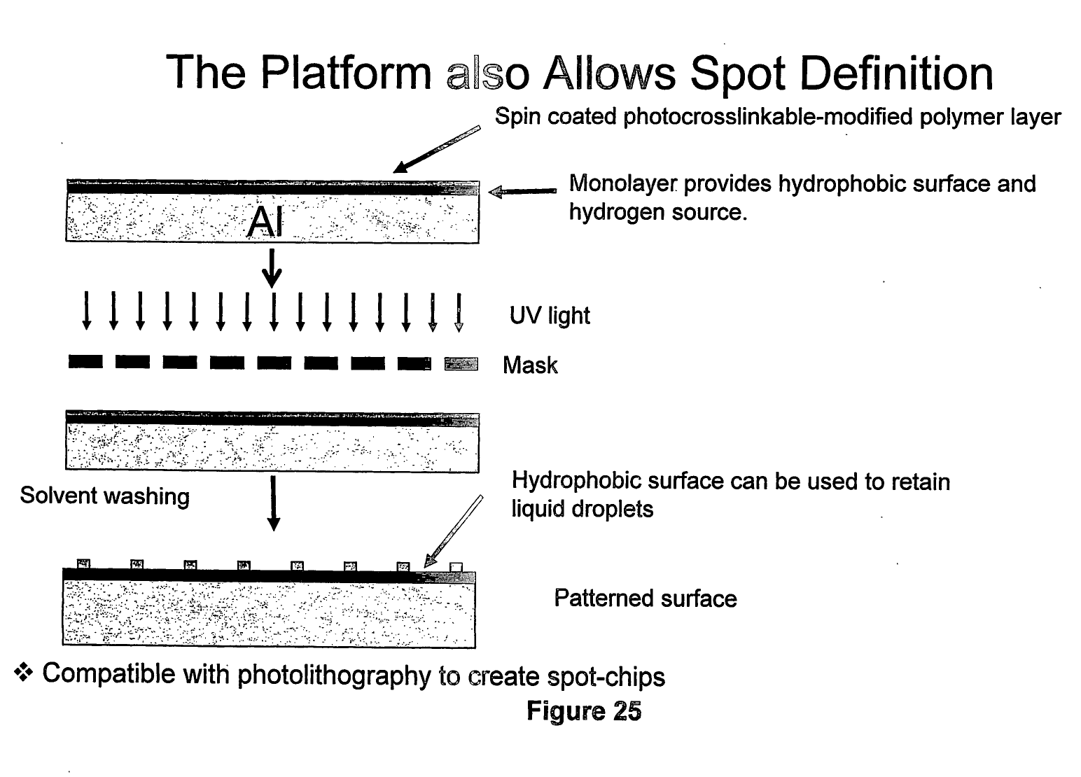

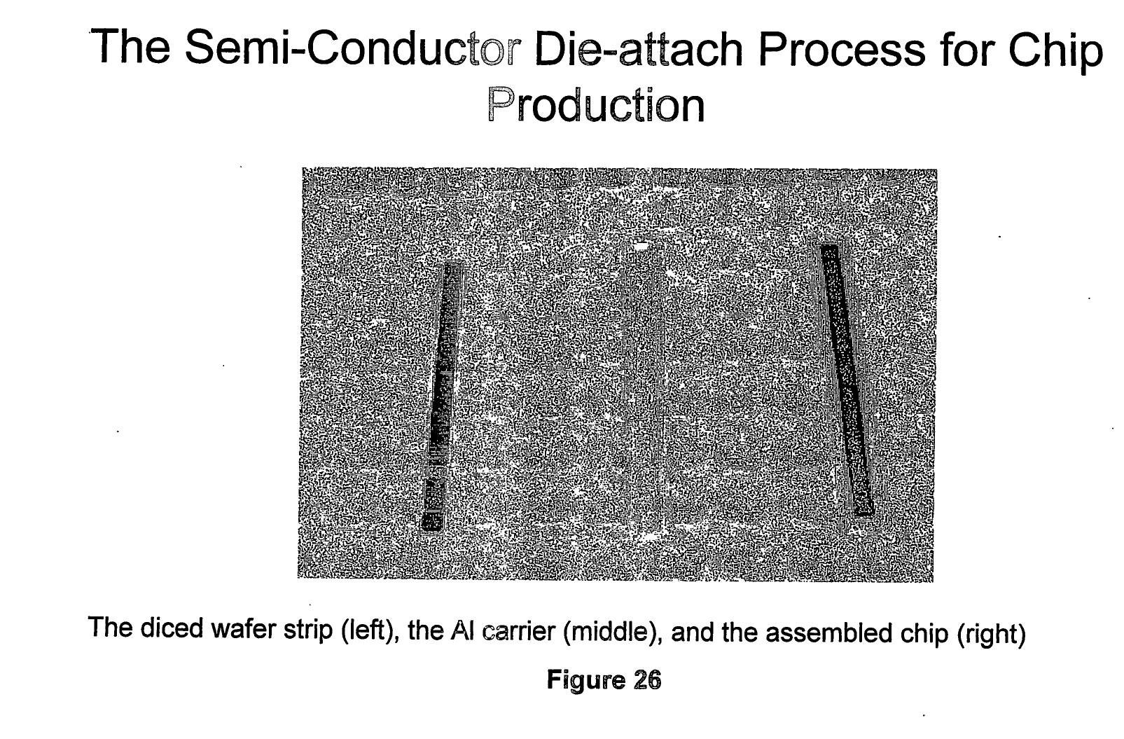

In addition to the above working examples, the present invention also can be practiced using the chemistries illustrated in FIGS. 21-23. In addition, the use of spin coating and of a photomask are illustrated in FIGS. 24 and 25, respectively. Use of a semiconductor die-attach approach is illustrated in FIG. 26.

The present invention provides novel materials and methods for analyzing biomolecular analytes in a sample. While specific examples have been provided, the above description is illustrative and not restrictive. Any one or more of the features of the previously described embodiments can be combined in any manner with one or more features of any other embodiments in the present invention. Furthermore, many variations of the invention will become apparent to those skilled in the art upon review of the specification. The scope of the invention should, therefore, be determined not with reference to the above description, but instead should be determined with reference to the appended claims along with their full scope of equivalents.

1. A copolymeric hydrogel precursor comprising:
(a) first monomeric subunits that comprise a photocrosslinkable functionality and
(b) second monomeric subunits that comprise a chemically selective functionality for binding a protein,
wherein the amounts of first and second monomeric subunits provide the copolymeric hydrogel precursor with the ability to be photocrosslinked into a hydrogel and the ability for the hydrogel to be selectively reactive with protein under aqueous conditions, whereby protein becomes bound to the chemically selective functionality.

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. A polymeric hydrogel precursor comprising photocrosslinkable functionality and chemically selective functionality, wherein the precursor is prepared by functionalizing a prefunctionalized polymeric hydrogel precursor with photocrosslinkable functionality and with chemically selective functionality, wherein the amounts of photocrosslinkable functionality and chemically selective functionality provide the hydrogel precursor with the ability to be photocrosslinked into the hydrogel and the ability for the hydrogel to be selectively reactive with protein under aqueous conditions, whereby protein becomes bound to the chemically selective functionality.

23. (canceled)

24. (canceled)

25. A copolymeric hydrogel precursor prepared by copolymerization of monomeric subunits comprising:
(a) first monomeric subunits that comprise a first free radical copolymerization functionality and a photocrosslinkable functionality and
(b) second monomeric subunits that comprise a second free radical copolymerization functionality and a chemically selective functionality for binding a protein,
wherein the amounts of first and second monomeric subunits provide, upon copolymerization, the polymeric hydrogel precursor with the ability to be photocrosslinked into the hydrogel and the ability to be selectively reactive with protein under aqueous conditions, whereby protein becomes bound to the chemically selective functionality.

26. A photocrosslinkable hydrogel precursor composition for selective interaction with protein under aqueous conditions consisting essentially of at least one hydrogel precursor polymer consisting essentially of (i) first comonomeric subunits that comprise a photocrosslinkable functionality and (ii) second comonomeric subunits that comprise a chemically selective functionality for interaction with a biomolecular analyte, wherein the photocrosslinkable hydrogel precursor composition is substantially free of photoinitiator.

27. A method for functionalizing a surface with copolymeric hydrogel, comprising:
(A) providing (i) a substrate presenting a surface and (ii) a copolymeric hydrogel precursor that comprises (a) first comonomeric subunits that comprise a photocrosslinkable functionality and (b) second comonomeric subunits that comprise a chemically selective functionality for binding a biomolecular analyte;
(B) contacting the copolymeric hydrogel precursor and the surface to form a layer of the copolymeric hydrogel precursor on the surface; and
(C) photocrosslinking at least some of the copolymeric hydrogel precursor layer to form hydrogel in contact with the surface.

28. (canceled)

29. (canceled)

30. (canceled)

31. (canceled)

32. (canceled)

33. (canceled)

34. (canceled)

35. (canceled)

36. A substrate that comprises a substrate surface and a hydrogel thereon, wherein the hydrogel comprises (i) a photocrosslinked functionality and (ii) a chemically selective functionality for binding a biomolecular analyte, wherein the hydrogel is substantially free of photoinitiator, and wherein the amount of the chemically selective functionality is sufficient for binding the biomolecular analyte.

37. The substrate according to claim 36, wherein the substrate surface is the surface of a primer layer.

38. The substrate according to claim 36, wherein the substrate surface is planar.

39. The substrate according to claim 38, wherein the substrate surface is the surface of a primer layer and wherein the hydrogel is a uniform layer.

40. The substrate according to claim 38, wherein the substrate surface is the surface of a primer layer, and the hydrogel is in the form of discreet spots.

41. The substrate according to claim 36, wherein the hydrogel is covalently bound to the substrate surface.

42. The substrate according to claim 41, wherein the hydrogel comprises a photocrosslinked benzophenone functionality.

43. The substrate according to claim 36, wherein the hydrogel is a uniform layer having a layer thickness of 10 nm to 10 microns.

44. The substrate according to claim 36, wherein the hydrogel is a uniform layer having a layer thickness of about 2 microns or less.

45. The substrate according to claim 36, wherein the hydrogel is in the form of discreet spots and has a spot thickness of 10 nm to 10 microns.

46. The substrate according to claim 36, wherein said substrate is a biochip and said hydrogel is covalently bound to the surface.

47. The substrate according to claim 46, wherein the substrate comprises a supporting layer that comprises a material selected from the group consisting of aluminum, silicon, glass, metal oxide, metal, polymer and composite, and wherein said surface is a surface of a primer layer that is supported on the supporting layer.

48. The substrate according to claim 46, wherein the substrate comprises a supporting layer that comprises polymer or composite.

49. The substrate according to claim 46, wherein the substrate comprises a supporting layer that comprises polymer and is electrically conductive.

50. The substrate according to claim 46, wherein the primer layer is a hydrophobic primer layer.

51. The substrate according to claim 50, wherein the primer layer is a silane primer layer, a hydrocarbon silane primer layer, a fluorinated silane primer layer, a mixed fluorinated/hydrocarbon silane primer layer, or a polymeric primer layer.

52. The substrate according to claim 37, wherein the primer layer is about 4 angstroms to about 3 microns thick.

53. The substrate according to claim 37, wherein the primer layer is about 4 angstroms to about 10 nm thick.

54. The substrate according to claim 47, wherein the hydrogel is present on the surface only in one or more discreet spots.

55. The substrate according to claim 54, wherein the hydrogel is present as a plurality of discreet spots, each having at least one lateral dimension that is about 100 nm to about 3 mm.

56. The substrate according to claim 55, wherein said lateral dimension is about 500 nm to about 500 microns.

57. The substrate according to claim 46, wherein the hydrogel is a uniform layer on the substrate surface having an average layer thickness of 5 nm to 10 microns.

58. The substrate according to claim 46, wherein the hydrogel is a copolymeric hydrogel, a dextran derivative, or a derivative of poly(2-hydroxyethyl methacrylate) or copolymer thereof.

59. The substrate according to claim 46, wherein the hydrogel is comprised of photocrosslinked benzophenone, diazo ester, aryl azide, or diazirine functionality.

60. The substrate according to claim 46, wherein said chemically selective functionality is a nucleophilic or electrophilic group.

61. The substrate according to claim 46, wherein said chemically selective functionality is an anionic or cationic group.

62. The substrate according to claim 46, wherein said chemically selective functionality is a carboxylic acid, amino, or quaternary amino group.

63. The substrate according to claim 46, wherein said hydrogel is a water-swellable polymer that comprises a linear, carbon backbone that has been crosslinked.

64. The substrate according to claim 46, wherein the hydrogel is a copolymer prepared by crosslinking of a precursor copolymer comprised of carboxylic acid-containing side groups and benzophenone-containing side groups.

65. The substrate according to claim 36, wherein the hydrogel is free of photoinitiator.

66. (canceled)

67. (canceled)

68. (canceled)

69. (canceled)

70. (canceled)

71. (canceled)

72. (canceled)

73. (canceled)

74. (canceled)

75. A copolymeric hydrogel precursor comprising:
(a) first monomeric subunits that comprise a photocrosslinkable functionality and
(b) second monomeric subunits that comprise a chemically selective functionality for binding a biopolymer,
wherein the amounts of first and second monomeric subunits provide the copolymeric hydrogel precursor with the ability to be photocrosslinked into the hydrogel and the ability for the hydrogel to be selectively reactive with biopolymer under aqueous conditions, whereby biopolymer becomes bound to the chemically selective functionality.

76. A copolymeric hydrogel precursor comprising:
(a) first monomeric subunits that comprise a photocrosslinkable functionality and
(b) second monomeric subunits that comprise a chemically selective functionality for interacting with a biopolymer,
wherein the amounts of first and second monomeric subunits provide the copolymeric hydrogel precursor with the ability to be photocrosslinked into the hydrogel and the ability for the hydrogel to be selectively interactive with biopolymer under aqueous conditions, whereby biopolymer becomes adsorbed to the chemically selective functionality.

77. A copolymeric hydrogel precursor comprising:
(a) first monomeric subunits that comprise a photocrosslinkable functionality, and
(b) third monomeric subunits that comprise an energy absorbing moiety.

78. (canceled)

79. (canceled)

80. (canceled)

81. (canceled)

82. (canceled)

83. (canceled)

84. (canceled)

85. (canceled)

86. (canceled)

87. (canceled)

88. (canceled)

89. (canceled)

90. (canceled)

91. (canceled)

92. (canceled)

93. (canceled)
